(19) United States
(12) Patent Application Publication
(10) Pub. No.: US 2021/0012899 A1
(43) Pub. Date: Jan. 14, 2021

(54) DIAGNOSIS FOR VARIOUS DISEASES USING TUMOR MICROENVIRONMENT ACTIVE PROTEINS

(71) Applicant: Otraces, Inc., Gaithersburg, MD (US)

(72) Inventors: Glaina Krasik, Montgomery Village, MD (US); Keith Lingenfelter, Potomac, MD (US)

(21) Appl. No.: 16/927,836

(22) Filed: Jul. 13, 2020

Related U.S. Application Data
(60) Provisional application No. 62/873,862, filed on Jul. 13, 2019.

(51) Int. Cl. G16H 50/20; G01N 33/68; G16H 10/60; G16H 10/40; G16H 50/70; G16H 70/60; G16H 50/50

(52) U.S. Cl. CPC G16H 50/20; G01N 33/6863; G01N 33/6869; G16H 50/50; G16H 10/40; G16H 50/70; G16H 70/60; G16H 10/60

(57) ABSTRACT

Systems and methods for disease diagnosis through the detection of multiple biomarkers by receiving concentration values of biomarkers, building a training set using the samples of the biomarkers, and performing correlation calculations on the biomarker concentration values to diagnose the disease.

### CROSS-REFERENCE TO RELATED APPLICATIONS

This application claims the benefit of U.S. Provisional Application No. 62/873,862, filed Jul. 13, 2019, the entirety of which is hereby incorporated by reference herein.

A related patent application, PCT/US2017/014595, (published as WO 2017/127822), filed Jul. 27, 2017, describes methods for improving disease prediction using an independent variable for the correlation analysis that is not the concentration of the measured analytes directly but a calculated value termed “Proximity Score” that is computed from the concentration but is also normalized for certain age (or other physiological parameters) to remove age drift and non-linearities in how the concentration values drift or shift with the physiological parameter (e.g., age, menopausal status, etc.) as the disease state shifts from not-disease to disease.

### FIELD OF THE INVENTION

The present invention relates to systems and methods for improving the accuracy of disease diagnosis and to associated diagnostic tests involving the correlation of measured analytes with binary outcomes (e.g., not-disease or disease), as well as higher-order outcomes (e.g., one of several phases of a disease). The focus of the described invention is detection of early stage cancer, specifically non-small cell lung cancer (NSCLC). The described invention is equally applicable to other solid tumor cancers, such as breast, ovarian, prostate cancers and melanoma.

The biomarkers discussed in the disclosure are primarily termed tumor microenvironment (TME) active proteins (cytokines). These biomarkers reveal actions and status of the tumor, as determined from noise suppressed serum blood measurements. Using methods disclosed in the referenced (above) patent application, real time tumor status and degree of aggressive growth of the tumor can be determined as described herein.

### BACKGROUND OF THE INVENTION

Diagnostic medicine has long held promise that proteomics, the measurement of multiple proteins with a correlation to the disease state, would yield breakthrough diagnostic methods in diseases for which research heretofore has not produced simple viable blood tests. Cancer and Alzheimer's are just two. A major problem has, in large part, boiled down to protein (or other biomolecule) concentration measurements of samples that are contaminated with factors related to other conditions or drugs (prescribed or not, e.g., alcohol), or that reflect geographic and environmental influences on biomolecule concentration measurements. Within a large population with known disease and not-disease states that would be used as the basis of a model to assess the correlation, there exists hundreds if not thousands of the conditions or drugs that affect up or down regulation of the biomarkers of choice. Furthermore, biological systems exhibit complex non-linear behaviors that are very difficult to model in a correlation method.

### SUMMARY OF THE INVENTION

The conventional wisdom in older proteomic methods is that the “truth” is in the raw concentration values measured, and their practitioners come from a biology or clinical chemistry background. In contrast, the methods of the present invention divert completely away from the notion that “truth” is in these raw concentration values and is based on a deeper interpretation of what the concentrations mean, as discussed below. These dramatically improve the performance of regression methods, the neural network solution, render the Support Vector Machine mute, and bring other more powerful correlation methods forward. The solution comes in part from the mathematics of measurements and rejection of random noise. All measurements consist of the desired signal and noise. Mathematics proves that the noise can be eliminated by multiple sampling of the desired signal. The noise will be separated by such sampling into correlated noise (in sync with the measurement sampling scheme) and uncorrelated or random noise. The random noise is reduced by the square root of the number of samples. The signal and correlated noise (called offset) can be deduced very accurately by this multiple sampling. Finally, the offset can be determined with measurements in the absence of signal. These methods are described and disclosed in detail in the referenced patent application, PCT/US2017/014595. The superior predictive power described for the TME active cytokines is produced by employing the methods described in that patent application.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1 is a graph which shows the Receiver Operator Characteristic (ROCD) Curve for the pro-inflammatory cytokine biomarker, IL 6, for 200 samples with and without diagnosed non-small cell lung cancer. This shows the TME signature behavior of the biomarker, as measured in noise suppressed serum;

FIG. 2 is a graph which shows the Receiver Operator Characteristic (ROC) Curve for the vascularization cytokine biomarker, VEGF, for 200 samples with and without diagnosed non-small cell lung cancer. This shows the TME signature behavior of the biomarker, as measured in noise suppressed serum;

FIG. 3 is a graph which shows the Receiver Operator Characteristic (ROCD) Curve for the tumor cell apoptosis cytokine receptor biomarker, TNF-Ri, for 200 samples with and without diagnosed non-small cell lung cancer. This shows the TME signature behavior of the biomarker, as measured in noise suppressed serum;

FIG. 4 is a graph which shows the Receiver Operator Characteristic (ROCD) Curve for the angiogenesis cytokine biomarker, IL 8, for 200 samples with and without diagnosed non-small cell lung cancer. This shows the TME signature behavior of the biomarker, as measured in noise suppressed serum;

FIG. 5 is a graph which shows the Receiver Operator Characteristic (ROCD) Curve for the Granular Colony Stimulating Factor, G-CSF cytokine biomarker, for 200 samples with and without diagnosed non-small cell lung cancer. This shows the TME signature behavior of the biomarker, as measured in noise suppressed serum;

FIG. 6 is a graph which shows the Receiver Operator Characteristic Composite Curve for Breast Cancer for all five Biomarkers VEGF, IL 6, PSA, IL 8 and TNFα. This shows amplification effect of the proteomic noise suppression and the spatial proximity correlation method, see referenced patent and the TME signature behavior of the biomarker, as measured in noise suppressed serum;

FIG. 7 is a graph which shows the action of the TME active biomarkers actions by NSCLC stage. This shows the modulation of these biomarkers as the tumor growth progresses;

FIG. 8A is a graph which shows the action of the TME active biomarkers actions by prostate cancer Gleason Score. This graph shows the modulation of these biomarkers as the tumor growth progresses;

FIG. 8B is a graph which shows the action of the TME active biomarkers actions by prostate cancer Gleason Score. This graph shows the modulation of these biomarkers as the tumor growth progresses;

FIG. 8C is a graph which shows the action of the TME active biomarkers actions by prostate cancer Gleason Score. This graph shows the modulation of these biomarkers as the tumor growth progresses;

FIG. 9 is a graph which shows two typical, IL 6 and VEGF, important biomarkers in 400 women that have been diagnosed with breast cancer or not;

FIG. 10 is a graph which shows the Proximity Score plot for the same two biomarkers for 400 women shown in FIG. 1 for IL 6 and VEGF;

FIG. 11 is a graph which shows the concentration to Proximity Score conversion for one equation set;

FIG. 12 is a graph which shows the concentration to Proximity Score conversion for another equation set;

FIG. 13 is a graph which shows the concentration to Proximity Score conversion for another equation set with zones folded over on top of another;

FIG. 14 are graphs which show the age distribution of the biomarkers PSA and TNFα mean concentration values;

FIG. 15 shows a 3D plot of IL 6 and VEGF Proximity Scores plotted on the horizontal axes and population distribution on the vertical axis;

FIG. 16 shows 3D plot of FIG. 15 with the horizontal axes rotated down showing the horizontal separation of the not cancer and cancer samples;

FIG. 17A is a graph which shows the ROC curves for CA 125, HE4 alone and the composite ROC curve for the ROMA test for ovarian cancer;

FIG. 17B is a graph which shows the ROC curves for CA 125, HE4 alone and the composite ROC curve for the ROMA test for ovarian cancer;

FIG. 17C is a graph which shows the ROC curves for CA 125, HE4 alone and the composite ROC curve for the ROMA test for ovarian cancer;

FIG. 18 is a 3D plot showing IL 6, VEGF and IL 8 plotted;

FIG. 19 shows the 3D plot in FIG. 18 rotated around the vertical axis and tilted back;

FIG. 20 shows the 3D plot in FIG. 18 rotated around to see the back through the origin;

FIG. 21 shows the 3D plot in FIG. 18 rotated upwards to show the cancer samples in front;

FIG. 22 is a graph which shows the actions on the five breast cancer biomarkers actions as the cancer progresses from healthy to stage 3 breast cancer;

FIG. 23 is a 3D plot of the biomarkers CA 125 and HE4 for ovarian cancer with population distribution of the Proximity Score shown on the vertical axis;

FIG. 24 shows the 3D plot of FIG. 23 rotated to show the population distribution of the HE4 biomarker more clearly;

FIG. 25 shows the 3D plot of FIG. 23 rotated down to show the two axes distribution of these two tumor marker more clearly;

FIG. 26 is a graph which shows the ROC curve for the breast cancer test discussed in this application;

FIG. 27 is a graph which shows population distribution for biomarker VEGF for 400 women diagnosed with and without breast cancer;

FIG. 28 is a graph which shows the concentration to Proximity Score conversion for one equation set;

FIG. 29 shows a task flow chart for the construction of the Training Set Model;

FIG. 30 is a graph which shows a stylized Proximity Score distribution with large non-linear distributions;

FIG. 31 is a graph which shows a stylized Proximity Score distribution with the large non-linear distributions suppressed;

FIG. 32 is a graph which shows a stylized Proximity Score distribution with a 50% to 50% disease to not disease distribution as required by the Training Set;

FIG. 33 is a graph which shows a stylized Proximity Score distribution with a disease to not disease true distribution;

FIG. 34 is a graph which shows a stylized Proximity Score distribution with a disease to not disease true distribution corrected by folding;

FIG. 35 is a graph which shows the resulting population distribution after conversion for biomarker VEGF;

FIG. 36 is a graph which shows the action of the TME active biomarkers actions by breast cancer. This shows the modulation of these biomarkers as the tumor growth progresses;

FIG. 37 is a graph which shows biomarker action by Gleason Score for prostate cancer;

FIG. 38 is a graph which shows biomarker action and cancer scores for breast cancer by stage; and

FIG. 39 shows an exemplary pathway by which the method of the present invention may be performed.

### DETAILED DESCRIPTION OF PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

The conventional wisdom in older proteomic methods is that the “truth” is in the raw concentration values measured, and their practitioners come from a biology or clinical chemistry background. In contrast, the methods of the present invention divert completely away from the notion that “truth” is in these raw concentration values and is based on a deeper interpretation of what the concentrations mean, as discussed below. These dramatically improve the performance of regression methods, the neural network solution, render the Support Vector Machine mute, and bring other more powerful correlation methods forward. The solution comes in part from the mathematics of measurements and rejection of random noise. All measurements consist of the desired signal and noise. Mathematics proves that the noise can be eliminated by multiple sampling of the desired signal. The noise will be separated by such sampling into correlated noise (in sync with the measurement sampling scheme) and uncorrelated or random noise. The random noise is reduced by the square root of the number of samples. The signal and correlated noise (called offset) can be deduced very accurately by this multiple sampling. Finally, the offset can be determined with measurements in the absence of signal. These methods are described and disclosed in detail in the referenced patent application, PCT/US2017/014595. The superior predictive power described for the TME active cytokines is produced by employing the methods described in this patent.

For the purposes of this application, specific terminology is used to better describe the preferred embodiments of the invention, which is defined below:

“Analytical Sensitivity” is defined as three standard deviations above the zero calibrator. Diagnostic representations are not considered accurate for concentrations below this level. Thus, clinically relevant concentrations below this level are not considered accurate and are not used for diagnostic purposes in the clinical lab.

“Baseline Analyte Measurement for an Individual” is a measurement set of the biomarkers of interest for the transition of an individual patient from the not disease state to the disease state, measured for a single individual multiple times over a period of time. The Baseline Analyte Measurement for the not disease state is measured when the individual patient does not have the disease, and alternatively, the Baseline Analyte Measurement for the disease state is determined when the individual patient has the disease. These baseline measurements are considered unique for the individual patient and may be helpful in diagnosing the transition from not disease to disease for that individual patient. The Baseline Analyte Measurement for the disease state may be useful for diagnosing the disease for the second or higher occurrence of the disease in that individual.

“Biological Sample” means tissue or bodily fluid, such as blood or plasma, that is drawn from a subject and from which the concentrations or levels of diagnostically informative analytes (also referred to as markers or biomarkers) may be determined.

“Biomarker” or “Marker” means a biological constituent of a subject's biological sample, which is typically a protein or metabolic analyte measured in a bodily fluid such as a blood serum protein. Examples include cytokines, tumor markers, and the like. The present invention also contemplates other indicia as “biomarkers” and “markers,” including but not limited to: height, eye color, geographic factor, environmental factors, etc. In general, such indicia will include any measurements or attributes that vary within a population and remain measurable, determinable, or observable.

“Blind Sample” is a biological sample drawn from a subject without a known diagnosis of a given disease, and for whom a prediction about the presence or absence of that disease is desired.

“Disease Related Functionality” is a characteristic of a biomarker that is either an action of the disease to continue or grow or is an action of the body to stop the disease from progressing. In the case of cancer, a tumor will act on the body by requesting blood circulation growth to survive and prosper, and the immune system will increase pro-inflammatory actions to kill the tumor. These biomarkers are in contrast to tumor markers that do not have Disease Related Functionality but are sloughed off into the circulatory system and thus can be measured. Examples of Functional Biomarkers would be Interleukin 6 which turns up the actions of the immune system, or VEGF which the tumor secretes to cause local blood vessel growth. Whereas a non-functional example would be CA 125. That is a structural protein located in the eye and human female reproductive tract and has no action by the body to kill the tumor or action by the tumor to help the tumor grow.

“Limit of Detection” (LOD) is defined as a concentration value 2 standard deviations above the value of the “zero” concentration calibrator. Usually the zero calibrator is run in 20 or more replicates to get an accurate representation of the standard deviation of the measurement. Concentration determinations below that level are considered as zero or not present for example, for a viral or bacterial detection. For purposes of the present invention, 1.5 standard deviations can be used when samples are run in duplicate, although the use of 20 replicates is preferred. Diagnostic representations requiring a single concentration number are generally not rendered below this level. Measurements at the level of Limit of Detection statistically are at a 95% confidence level. Predictions of disease state using the methods discussed here are not based upon a single concentration and predictions are shown to be possible at measurements levels below the concentration based LOD.

“Low Abundance Proteins” are proteins in serum at very low levels. The definition of this level is not clearly defined in the literature but as used in this specification, the level would be less than about 1 picogram/milliliter in blood serum or plasma and other body fluids from which samples are drawn.

“Meta-variable” means information that is characteristic of a given subject, other than the concentrations or levels of analytes and biomarkers, but which is not necessarily individualized or unique to that subject. Examples of such meta-variables include, but are not limited to, a subject's age, menopausal status (pre-, peri- and post-) and other conditions and characteristics such as pubescence, body mass, geographic location or region of the patient's residence, geographic source of the biological sample, body fat percent, age, race or racial mix, or era of time.

“Population Distribution” means the range of concentrations of a particular analyte in the biological samples of a given population of subjects. A specific “population” means but is not limited to: individuals selected from a geographic region, a particular race, or a particular gender. And the population distribution characteristic selected for use as described in this application further contemplates the use of two distinct subpopulations within that larger defined population, which are members of the population who have been diagnosed as having a given disease state (disease subpopulation) and not having the disease state (non-disease subpopulation). The population can be whatever group in which a disease prediction is desired. Moreover, it is contemplated that appropriate populations include those subjects having a disease that has advanced to a particular clinical stage relative to other stages of disease progression.

“Population Distribution Characteristics” are determinable within the population distribution of a biomarker, such as the mean value of concentration of a particular analyte, or its median concentration value, or the dynamic range of concentration, or how the population distribution falls into groups that are recognizable as distinct peaks as the degree of up or down regulation of various biomarkers and meta-variables of interest are affected by the onset and progression of a disease as a patient experiences a biological transition or progression from the non-disease to disease state.

“Predictive Power” means the average of sensitivity and specificity for a diagnostic assay or test, or one minus the total number of erroneous predictions (both false negative and false positive) divided by the total number of samples.

“Proximity Score” means a substitute or replacement value for the concentration of a measured biomarker and is, in effect, a new independent variable that can be used in a diagnostic correlation analysis. The Proximity Score is related to and computed from the concentration of measured biomarker analytes, where such analytes have a predictive power for a given disease state. The Proximity Score is computed using a meta-variable adjusted population distribution characteristic of interest to transform the actual measured concentration of the predictive biomarker for a given patient for whom a diagnosis is desired, as disclosed in International Publication No. WO 2017/127822 and International Publication No. WO 2014/158287. “Proximity Score” and “pseudo-concentration” have the same definition and may be used interchangeably.

“Slicing the Multi-Dimensional Grid” is useful for reducing the computation time needed to build the model. In this case, the multi-dimensional space, 5 dimensions, is cut into 2 dimensional slices along each set of orthogonal axes. This yields 10 “bi-marker planes” for the 5-dimensional case (6 dimensions would yield 15 planes). The training set data is then plotted on each plane, and the planes are again cut up into grid sections on each axis. Each bi-marker plane is thus a projection of the full multi-dimensional grid on the bi-plane.

“Proteomic Mean Value Separation” determines if the biomarkers of interest can actually separate the two conditions of interest signal (disease) or Null Offset (not-disease). If the mean values are measured accurately in a known population and they have separation (are different in value), then diagnostic predictive power will be achieved.

“Proteomic Noise Suppression” is the method whereby the aforementioned Proteomic Variance (noise) is suppressed. This suppression is done first on the known group of samples, termed the training set. The goal is to condition the concentration values of the training set samples such that they agree with the medically determined diagnosis. The mathematical methods are limited only by the goal of forcing the predictive scoring of the predictive model to agree with the known samples. The method may involve compression, expansion, inversion, reversal, folding portions of measured variables over onto itself producing a function where multiple inputs (concentrations) produce the same output (Proximity Score). The reasons for this are several (see below population distribution bias) and include the purpose of damping the variance “noise.” Also, look up tables or similar tools can be used for the transformation, and for other mathematical schemes. This same noise suppression method, when applied to blind or validation sample, will produce this same noise suppression. The result after the transformation is called the Proximity Score. Suppression of proteomics variance is the mathematical transformation that eliminates or suppresses the variation not correlated with the conditions of interest, in this case not-breast cancer and breast cancer defined by the mean values of both as measured in a large known population of each.

“Specificity” is a true false positive rate of a test. It is mathematically one minus the false positive number of measurements of the test divided by the total number of true negative samples measured.

“Incongruent Training Set Model” (or “Secondary Algorithm”) is a secondary training set model that uses a different phenomenological data reduction method such that individual points on the grids of the bi-marker planes are not likely to be unstable in both the primary correlation training set model and this secondary algorithm.

“Spatial Proximity Correlation Method” (or Neighborhood Search or Cluster Analysis) is a method for determining a correlation relationship between independent variables and a binary outcome where the independent variables are plotted on orthogonal axes. The prediction for blind samples is based upon proximity to a number (3, 4, 5 or more) of so called “Training Set” data points where the outcome is known. The binary outcome scoring is based upon the total distance computed from the blind point on the multi-dimensional grid to Training Set points showing the opposite outcome. The shortest distance determines the scoring of the individual blind data point. This same analysis can be done on bi-marker planes cut through the multidimensional grid where the individual bi-marker plane score is combined with the score of the other planes to yield a total. This use of cuts of the two-dimensional orthogonal projections through the space can reduce computation time.

“Training Set” is a group of patients (200 or more, typically, to achieve statistical significance) with known biomarker concentrations, known meta-variable values and known diagnosis. The training set is used to determine the axes values “Proximity Scores” of the “bi-marker” planes as well as score grid points from the Spatial Proximity analysis that will be used to score individual blind samples.

“Training Set Model” is an algorithm or group of algorithms constructed from the training set that allows assessment of blind samples regarding the predictive outcome as to the probability that a subject (or patient) has a disease or does not have the disease. The “training set model” is then used to compute the scores for blind samples for clinical and diagnostic purposes. For that purpose, a score is provided over an arbitrary range that indicates percent likelihood of disease or not-disease or some other predetermined indicator readout preferred by a healthcare provider who is developing a diagnosis for a patient.

“Orthogonal” is a term used in this description of the method that applies to low level signaling functions such as adaptor, effecter, messenger, modulator proteins, and the like. These proteins have functions that are specific to a body's reaction to the disease or the disease's action on the body. In the case of cancer, these are generally considered to be immune system actors such as inflammatory, or cell apoptosis and vascularization functions. One tumor marker is considered to be orthogonal to the extent that it does not also represent a specific signaling function. The marker should be selected as best as possible to be independent of the others. In other words, varying levels on one should not interact with the others except as the disease itself affects both. Thus, if variations in one orthogonal function occur, these changes in and of themselves will not drive changes in the others. Vascularization and inflammatory functions would be considered orthogonal in that proteins can be selected that primarily perform only one of these functions. These proteins, when plotted on the multi-dimensional Spatial Proximity grid, will act independently, and if the disease causes actions of both, they will amplify predictive power. Many cytokines have multiple interacting functions, thus the task is to select functions and the proteins such that this interaction is limited. The degree of “functional orthogonality” is a relative matter, and in fact it can be argued that all cytokines interact to some degree. Many have severely overlapping functions and many do not. Interleukin 8 is implicated in both pro and anti-inflammatory actions as well as angiogenesis. In a disease such as cancer, it is primarily the circulatory action, but other existing conditions within the organism may well be driving actions of this cytokine, contributing to the Proteomic Variance. The choice of best biomarkers with functional orthogonality is at best a compromise depending on the conditions being diagnosed.

“Receiver Operator Characteristic (ROC) curve” is a graphical method for representing the performance of a signaling method used for decision making where there is a tradeoff between the false positive, false negative rates and the intensity of the detecting signal. In this graphical representation, the ordinate of the plot contains the sensitivity of the test method, and the abscissa has the false positive rate. For biomarkers (or signals) with upward action to the disease trip point, the curve will be above a 45° null line originating at the origin (0,0) of the plot to the upper right of the plot (1.0,1.0). The area under the curve indicates how good the biomarker is at making the prediction.

“ROC Curve ‘Area Under the Curve’ (AUC)” is the area under the biomarker characteristic curve and the abscissa. For a perfectly useless biomarker, the AUC will be 0.5 and is the area under the 45° null line referred to above. A perfect test has an AUC of 1.0 and extends from the origin up the ordinate to the 100% sensitivity point and then across the ROC curve to the 1.0, 1.0 point at the upper right.

“Tumor Microenvironment” is bathed in the tumor interstitial fluid (TIF), is the cellular environment in which the tumor exists, including surrounding blood vessels, immune cells, fibroblasts, bone marrow-derived inflammatory cells, Lymphocytes, signaling molecules and the extracellular matrix.

“Tumor Marker” is a protein marker that is sloughed off into the TME or blood supply that has no apparent function, is either the tumor's growth by tumor secretions or the tumor's suppression by the immune system.

These methods involved determining the mean values of the biomarkers for the defined populations for the conditions to be predicted, e.g. cancer vs. not cancer or cancer stage, and suppressing the raw concentration measurements anchored by these mean values. Also, the drift in mean concentration by age, or other metavariable, selected must be normalized or zeroed out in the transition to a new correlation independent variable termed proximity score. This new set of independent variables in then used in the correlation to the prediction of the disease state.

### Tumor Microenvironment Biomarkers

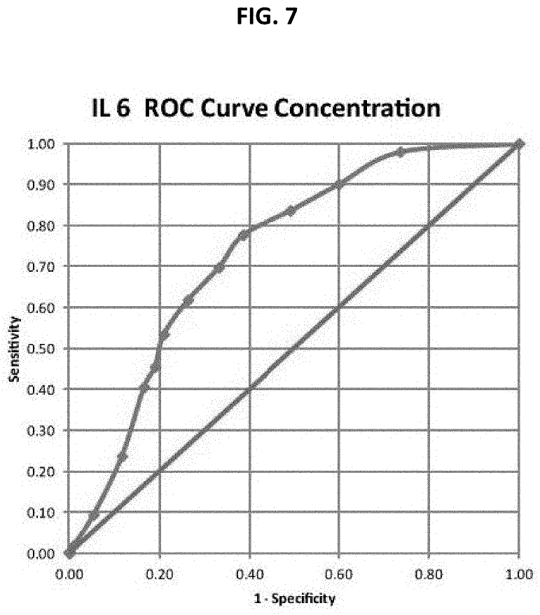

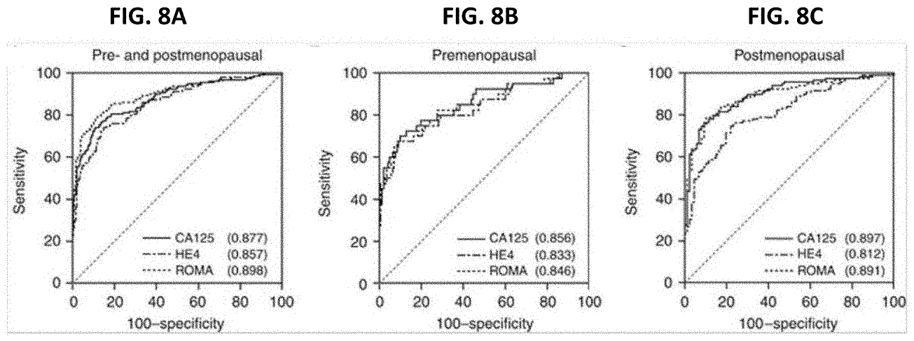

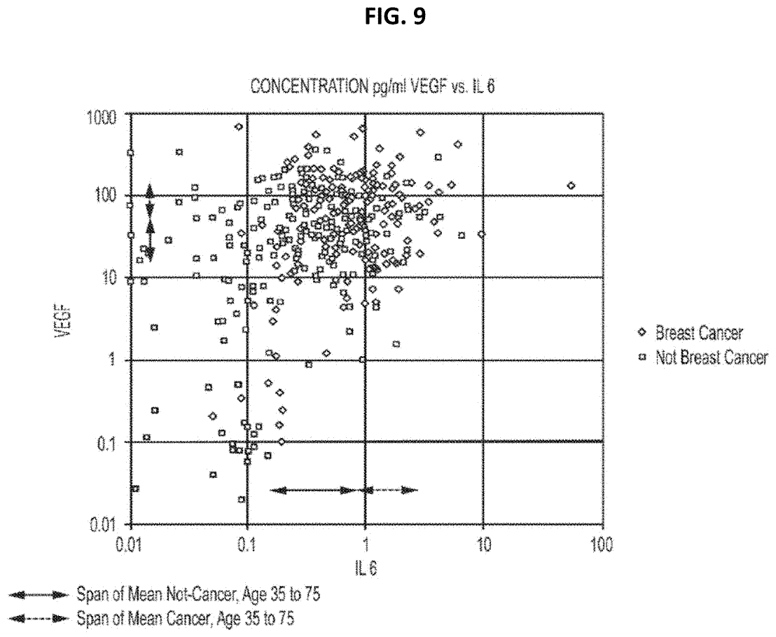

Noise suppressed serum biomarkers can be used to determine the signature of the actions of the tumor and the immune system within the TME. These actions include actions by the tumor to suppress the tumor growth, pro-inflammatory cytokines and anti-tumor or apoptosis cytokines. Also included are actions by the tumor to grow, including angiogenesis, blood vessel growth in surrounding tissue and vascularization and blood vessel growth within the tumor bulk. Also, actions by the tumor to suppress the immune system, where anti-inflammatory cytokines are important. The actions of these biomarkers expose the status and behavior of the tumor as a snapshot in time frozen at the instant of blood draw. FIGS. 7 and 8A-C show these actions by cancer stage for NSCLC and prostate cancer and FIG. 9 for breast cancer. Generalized comments can be made about this behavior as the tumor progresses from the healthy to the malignant state and through various cancer stages. This behavior is also indicative of other solid tumor cancers such as ovarian.

At the onset of an early stage, nascent tumor, the immune system responds strongly. The biomarkers for pro-inflammatory and tumor apoptosis responded strongly. Also typically seen is a strong response by the tumor for stimulating blood vessel growth in the surrounding tissue. As the tumor progresses, it secretes anti-inflammatory cytokines suppressing the immune system. As the tumor bulk increases, a strong up regulation in tumor secretion of vascularization cytokines is seen. These combined actions, when properly noise suppressed in serum measurements, show the tumor and immune system actions and the detailed status on the tumor.

### Specific Cytokines—Pro-Inflammatory

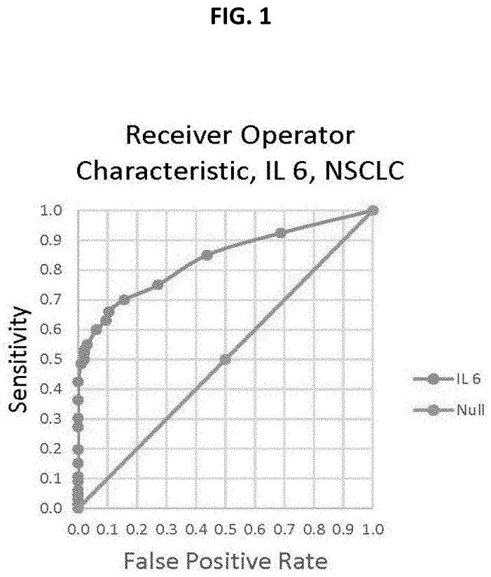

Generally, interleukin 6 has been found to be probative for this immune system action, however, others are possible important actors; interleukin 1, interleukin 1β, IL-12, and IL-18 are others. The Receiver Operator Characteristic Curve for IL 6 for NSCLC is shown in FIG. 1. This biomarker alone cannot adequately detect the presence of NSCLC. At 90% sensitivity, the false positive rate is fairly high at about 60%.

### Specific Cytokines—Tumor Vascularization

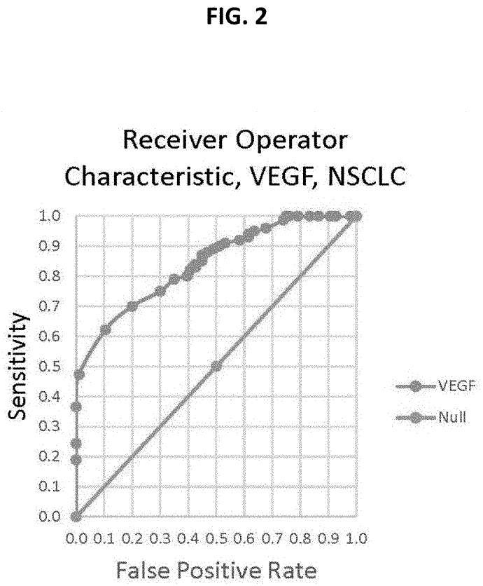

Bulk tumor vascularization is associated primarily with vascular endothelial growth factor, VEGFβ. Other cytokines in this functional group may be Placental Growth Factor (PLGF), VEGF-A, VEGF-C and VEGF-D: VEGF-A binds to VEGFR1 and VEGFR2. The Receiver Operator Characteristic Curve for VEGF for NSCLC is shown in FIG. 2. This biomarker alone cannot adequately detect the presence of NSCLC. At 90% sensitivity the false positive rate is fairly high at about 50%.

### Specific Cytokines—Tumor Directed Cell Apoptosis

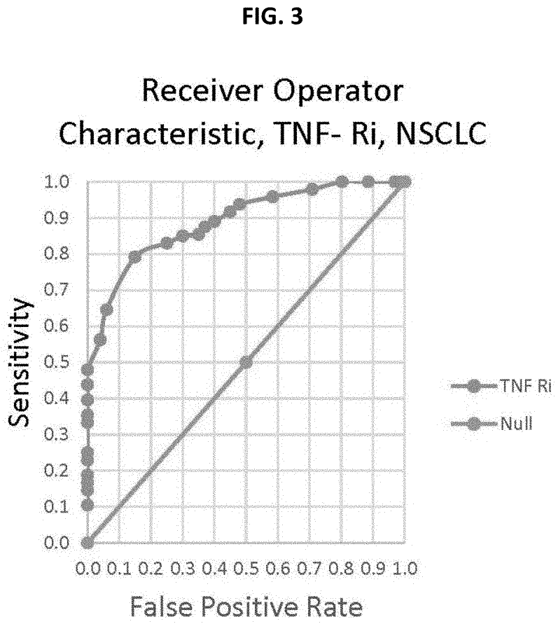

Cytokines in the tumor necrosis family perform a number of immune system functions, ranging from inflammation to T and B cell regulation, through inhibition of angiogenesis. Certain cytokines in the family are focused on cell apoptosis, programmed cell death. These are TNFα, CD254, DR3L, CD258 and TNA receptors (1 and 2). The Receiver Operator Characteristic Curve for TNF Ri for NSCLC is shown in FIG. 3. This biomarker alone cannot adequately detect the presence of NSCLC. At 90% sensitivity, the false positive rate is fairly high at about 45%.

### Specific Cytokines—Tumor Angiogenesis

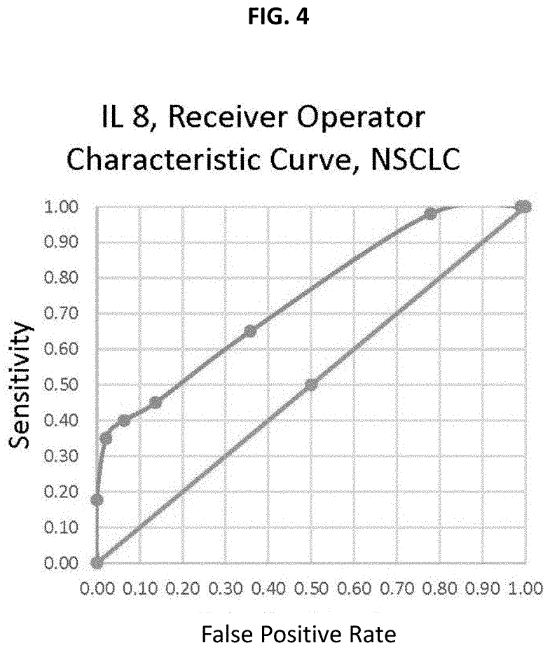

Angiogenesis is associated with vascularization, however, in this context the focus is on stimulation of blood vessel growth at tumor early stage in the immediate surrounding tissue. Interleukin 8 is associated with this. The Receiver Operator Characteristic Curve for IL 8 for NSCLC is shown in FIG. 4. This biomarker alone cannot adequately detect the presence of NSCLC. At 90% sensitivity, the false positive rate is fairly high at about 65%.

### Specific Cytokines—Colony Stimulating Factors

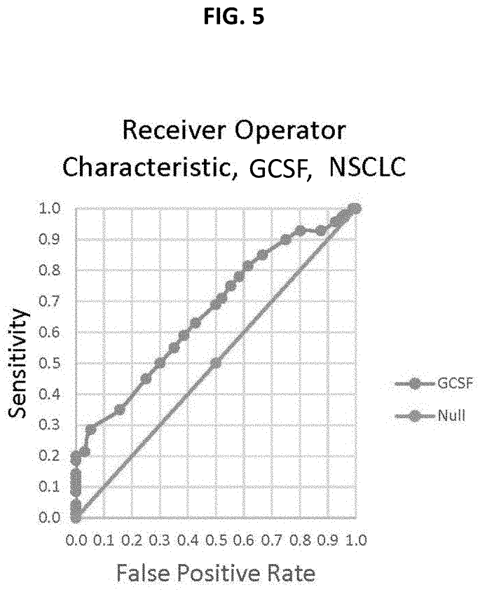

These cytokines seem to be implicated in initiation of angiogenesis and vascularization and are secreted by the tumor. Primary factors are granulocyte stimulating factor G-CSF, but also implicated are granular macrophage stimulating factor GM-CSF, and macrophage stimulating factor GSF. The Receiver Operator Characteristic Curve for G-CSF for NSCLC is shown in FIG. 5. This biomarker alone cannot adequately detect the presence of NSCLC. At 90% sensitivity, the false positive rate is fairly high at about 75%.

### Combining Biomarkers With Proteomic Noise Suppression

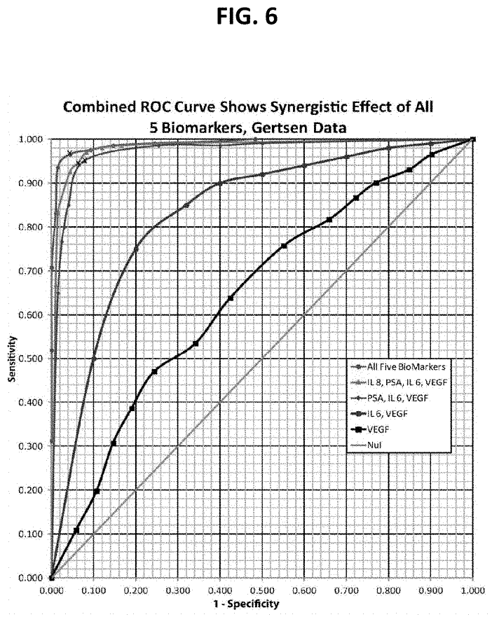

These TME active cytokines cannot each alone accurately predict the presence of NSCLC. The contamination from serum based actions from other conditions that may be present creates “noise” that reduces specificity. By employing noise suppression methods as described in the referenced PCT/US2017/014595 patent application, these problems can be mitigated. The example outlined in the referenced patent application for breast cancer shows how the method allows this to work. The example used proteins from similar TME active functional groups, and graphically shows the dramatic improvement in predictive power achieved. The example is repeated here (see FIGS. 6, 7 and 8A-C). FIG. 6 shows the combined ROC for each of the five biomarkers used a in similar breast cancer test panel for detecting the presence of this cancer. FIG. 7 shows the ROC curve for the biomarker IL 6 for breast cancer. The IL 6 ROC curve shows it achieves a poor 60% false positive rate at 90% sensitivity. In FIG. 6, the standalone ROC for VEGF is shown with a very poor false positive rate of 78% of again 90% sensitivity.

When these two biomarkers are combined using the proteomic noise suppression method and the spatial proximity correlation, these two biomarkers achieve a 40% false positive rate at 90% sensitivity. A detailed description of this is found in the referenced PCT/US2017/014595 patent application.

The method in part depends on using what are termed functionally orthogonal proteins that are TME active. These proteins are noise-suppressed, plotted, and scored in multi-dimensional space, as they up-regulate in the transition to disease.

Standard correlation methods cannot achieve this as they cannot trap spatial separation vectors produced by the noise suppressed concentration information. That is shown graphically in FIGS. 8A-C. These ROC curves are for the Abbott ROMA test that uses two tumor markers HE4 and CA 125 to recommend a treatment vector for ovarian cancer. Note the two standalone biomarkers are similar in ROC curve performance with about a 35% to 45% false positive rate at 90% sensitivity. Note also that the combined ROC is no better than either single tumor marker alone. That is because simpler correlation methods such as logistic regression, neural networks and ROC curve area enhancements methods cannot trap spatial separation information.

This combined biomarker set, as shown in FIGS. 8A-C, achieves 99% specificity and 97% sensitivity. The breast cancer test panel discussed above using these methods achieves 96% sensitivity and 97% sensitivity.

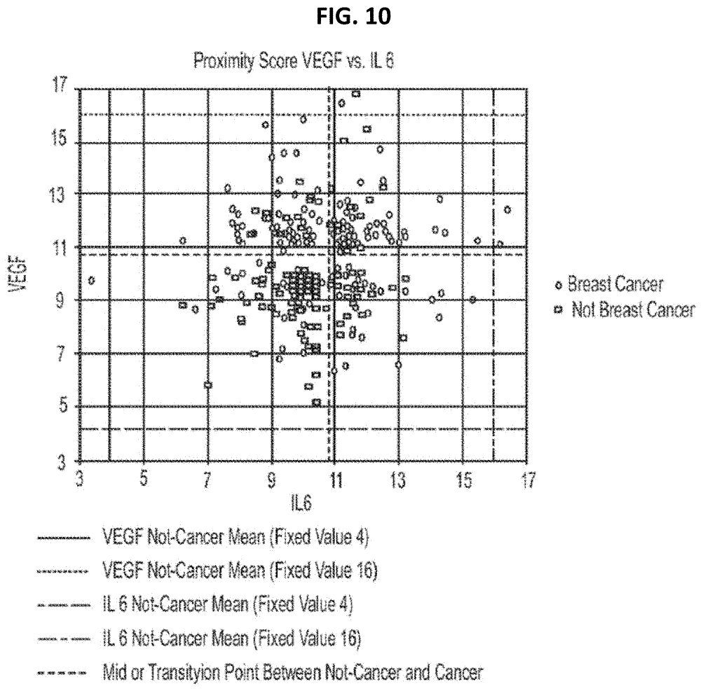

The presence of these conditions is in general unknown in patients seeking screening for a specific disease, (e.g., breast cancer), and the question asked is in which group does the unknown patient fit in, the not-breast cancer or the breast cancer group. The unknown variance must be dampened as it is done in Proteomic Variance, “noise” suppression in the measurement science, in order to answer this question. Note that both the breast cancer positive patients and the not-breast cancer concentration measurements are contaminated with this extraneous information. Furthermore, the notion of the “proper” value for these biomarkers for a “healthy” individual as well as an individual with the disease is meaningless. The only way to make sense of this scattering of the concentration data is to dramatically suppress the noise for both of the cohorts by anchoring on the mean values and suppressing all other information in the concentration data. The result is the Proximity Score. One could say that the notion of “proper values” for these concentrations for a “healthy” or diseased individual is meaningless. The extraneous information, Proteomics variance “noise”, is what contributes to the scatter in FIG. 9. This noise suppression is what produces the cleaner plot in FIG. 10.

The first step is to reconcile what can be known about the FIG. 9 plot for breast cancer. There are limited pieces of information in the plot that relate to the question: is the unknown patient likely to have a not-breast cancer disease state or a breast cancer disease state. The information in the plot are the mean values of the two biomarkers for both not-breast cancer and breast cancer. Beyond these mean values, we can rank each individual sample by its relationship to the means. There are only four ranks or zones: 1) the individual sample is less than the mean value for not-breast cancer; 2) the individual sample is greater that this mean value for not-breast cancer but less than the derived mid-point mean value between the breast cancer/not-breast cancer means; 3) the individual sample is above this midpoint of the means and below the mean value for cancer; and 4) the individual sample is above the mean value for breast cancer. Furthermore, the mean values noted for each state and each biomarker drift with age. Thus, the relationship between age and the mean values must be known. Each of the rankings noted above must be limited for any one patient to the mean for that patient's age. Any information beyond this for individual samples is not useful and can be considered Proteomic Variance (noise). These five pieces of information (age and relationships of the means and midpoint) are the deeper interpretation of the raw concentration measurements. As noted, this information, when evaluated according to the present invention, surprisingly reflects the truth with respect to the question at hand, is the patient not-disease or disease. And thereby provides a method of indicating the probability of a disease state existing in a patient under examination.

Finally, the mean values and ranking are transferred from the raw concentration such that the mean values are normalized and the noted ranks are plotted in specific zones. This transformation from raw concentration, anchored by age adjusted means and age adjusted rankings with respect to the means, produces a new independent variable for the Spatial Proximity plot and correlation method. This variable is called a Proximity Score.

FIG. 10, as discussed above, shows the resultant bi-plane plot after conditioning the raw concentration into Proximity Score. Also, the age drift is normalized such that all age groups are positioned at a fixed or set point for each biomarker. Thus, if an unknown patient sample happens to have a concentration value at the not-cancer mean value for its age, then its Proximity Score will be fixed at the set value, and all patient samples at all ages who are at the mean value will get that same value in Proximity Score.

In this example, the set values are arbitrarily set at 4 for not-cancer mean and 16 for cancer mean. Other values could be used, such as a broader range, for example. Also, note that in this example the raw outlying concentration values achieve best fit to the known patient diagnosis of the training set by folding these concentrations into the space between the now newly set fixed mean values for pseudo-concentration. This achieves the damping of noise needed and the transformation is designed to retain the clumping behavior that the correlation method is based upon, the Spatial Proximity Correlation.

Each individual raw concentration value is then placed within one of 4 “ranks” based upon its position with respect to the means at its age in the concentration space. Once converted to Proximity Score, age is removed from the new independent variable for the correlation (see below for details). This is not the only equation set for this task and best fit of the training set to the real diagnosis. The design of this transformation is based upon the fundamental characteristics of the raw data to be fitted and the underlying characteristics of the Spatial Proximity method. A workable solution can be found by iterative trials.

Use of these five biomarkers described in this application, IL 6, IL 8, VEGF, TNFα, and PSA for breast cancer, and yields the predictive power noted in Table 2 above for various correlation methods. While these particular markers are sufficiently orthogonal and provide sufficient information to separate disease states, it is contemplated by the inventors that other sets of biomarkers can be utilized and different numbers of biomarkers in such sets may vary.

These biomarkers produce predictive power with standard logistic regression methods typical of any group of five such markers. This level of predictive power is also typical of the various Receiver Operator Characteristic (ROC) curve methods for maximizing the aggregate area under the ROC curve (i.e., about 80%). The conversion to logarithm scales is also typical as the raw concentration ranges often exceed 5 orders of magnitude. Also, using the logarithm of concentration with the Support Vector Machine and Spatial Proximity correlation method yields better predicative power (i.e., 84 to 85%). This is likely due to the spatial separation effects of these biomarkers. The conversion to Proximity Score (reduction in extraneous information) also yields even more significant improvement in predictive power (i.e., 87 to 90%). However, the best predictive power results with the combination of all three, these functionally orthogonal biomarkers, Spatial Proximity correlation, and the conversion to Proximity Score (i.e., 96%). Finally, correcting the Spatial Proximity method for topology instability improves this predictive power to greater than 96%.

The analytical model comprising an embodiment of the methods of the present invention generally follows the following steps:

1) Collect a large group of known not-disease and disease patient samples. They should not be screened for any other unrelated conditions (non-malignant for cancer) but collected such that they look statistically like the general population.

2) Measure the biomarker parameter concentrations.

3) Compute the mean values of these biomarkers for the not-disease and disease group (see additional considerations below under age drift of the mean values).

4) Mathematically manipulate the raw concentrations to force them into groupings that mimic the mean values. This may involve compression, expansion, inversion, reversal, look up tables for transformation, and other mathematical operations. The method may contain some or all of these schemas. The resulting numerical value may not resemble the original concentration values at all, and one may not be able to work back from the resulting value to concentration as the transformation curve may fold back on itself. This new independent variable for the correlation is called Proximity Score. In fact, the resulting distribution is likely to be piled up near the two mean values with the mean value anchor points retained.

5) The manipulation also must force the unknown sample into rankings based upon that sample's relationship to the aforementioned mean values. Herein, we define zones that are respectively: 1) below the unknown sample's mean value at its age for not-disease; 2) above the not-disease mean value at its age but below the derived midpoint between the not-disease mean and disease mean at its age; 3) above the derived midpoint between the not-disease mean and disease mean but below the disease mean value at its age; and 4) above the unknown sample's mean value at its age for disease. These zones can be compressed into spaces near and/or on the respective means to dampen variances caused by the unrelated contaminating conditions or drugs.

6) The aforementioned mean values must take into account the age of each patient who contributes a biological sample. The zone positioning of each sample must be related to the corresponding patient's age and the mean values of the disease and not-disease means at that patient's age.

7) Possible Equations Used for Concentration to Proximity Score Conversion

The Ratio Log Linear Equation Used for OTraces Breast and prostate Cancer Determination is:

One equation for conversion of concentration to Proximity Score discussed in the referred application is:

PSₕ=K*logarithm₁₀((Ci/C₍ₕ))−(Cc/Ch))+Offset Equation 1

PS_{c}=K*logarithm₁₀((Ci/Cc)−(Ch/Cc))²+Offset Equation 2

Where:

PSh=Proximity Score for not-cancer

PSc=Proximity Score for cancer

K=gain factor to set arbitrary range

Ci=measured concentration of the actual patient's analyte

Ch=patient age adjusted mean concentration of non-disease patients' analyte

Cc=patient age adjusted mean concentration of disease patients' analyte.

Offset=Ordinate offset to set numerical range (arbitrary)

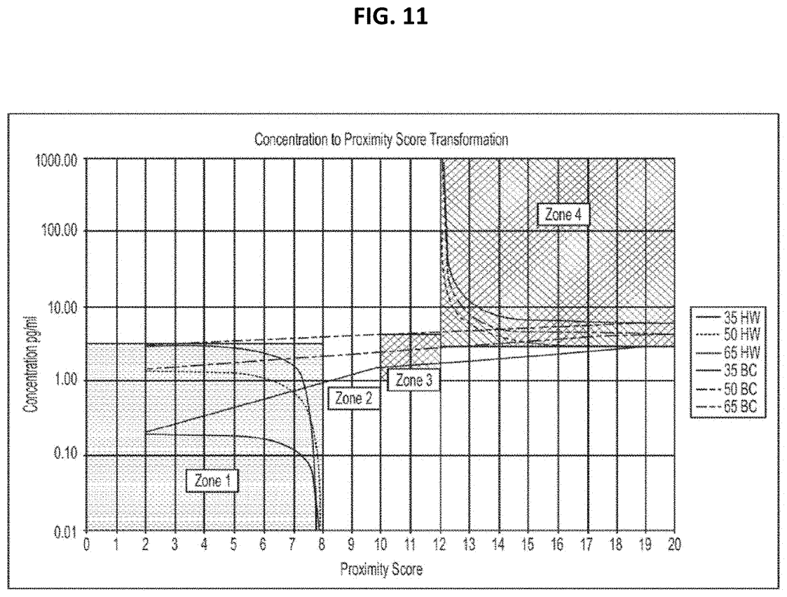

This embodiment, FIG. 11, shows Zone 1 fold on to Zone 2 and Zone 4 folded back on Zone 3 (see section on Population Distribution Bias). In the case of Cancer Versus not Cancer the cancer cohort is over represented in the training set by a large margin. The folding improves the distribution bias the zones dominated by not cancer. This embodiment is shown in the figure.

8) Another Embodiment uses straight log concentration to linear conversions.

where:

PS=M(log(Ci)+B

and PS=Proximity Score the concentration

Ci=measured concentration of the actual patient's analyte

M=conversion slope

B=Offset

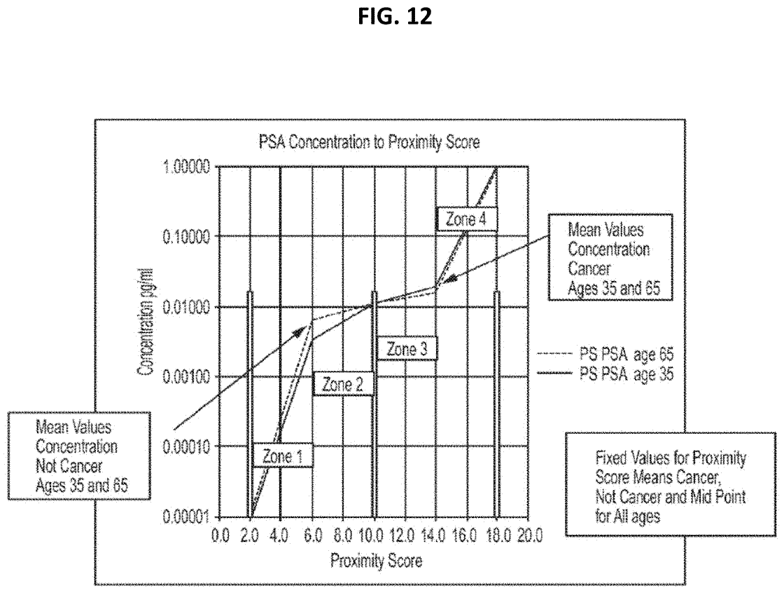

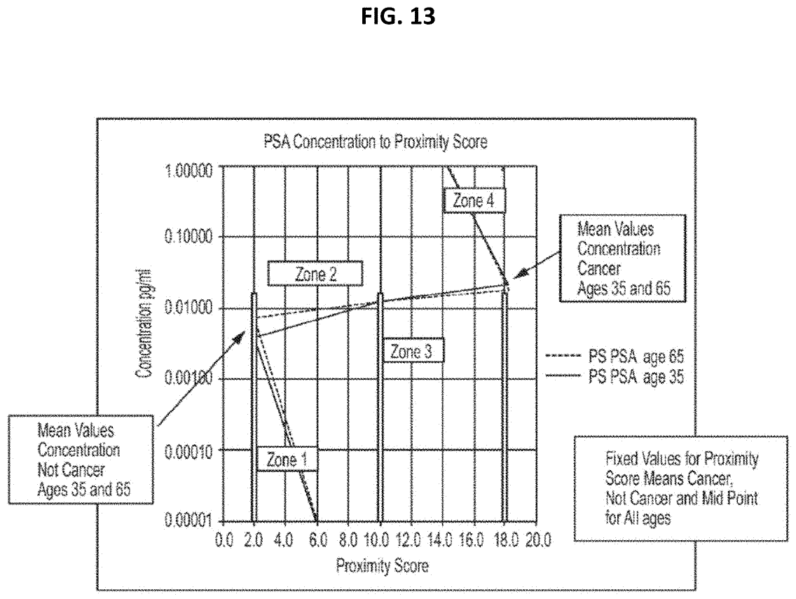

This embodiment is shown in FIGS. 12 and 13. FIG. 12 shows the order of the four zones in maintained order on the Proximity Score axis. FIG. 13 shows the zones 1 and 2 overlapped as are zone 3 and 4 (see population distribution bias below). Folding Zone 1 folded on to Zone 2 and Zone 4 folded back on Zone 3 is useful where the population distribution of the two states “A” and Not “A” are somewhat equal in population distribution.

7) This new variable called Proximity Score is applied to the correlation method of choice (see sections herein for discussions of this). 8) Using the same schema as developed to maximize predictive power within the training set model, determine whether an unknown samples “fits” either in the not-disease or disease group.

The age related mean value function is the anchor point for the transition from raw concentration and the new Proximity Score used in the correlation on the Spatial Proximity Grid. This function is determined from a large population of known disease and not-disease samples, and the population can include the training set but can also include a larger group. The not-disease and disease populations are defined as noted below. It is a function that relates mean value of not-disease and disease to age as it drifts. It is used to place the mean values to fixed positions on the Proximity Score axis where raw concentration is converted to Proximity Score. It will usually result in a family of equations that perform the transformation—one for each year of age. This function allows normalization of age drift.

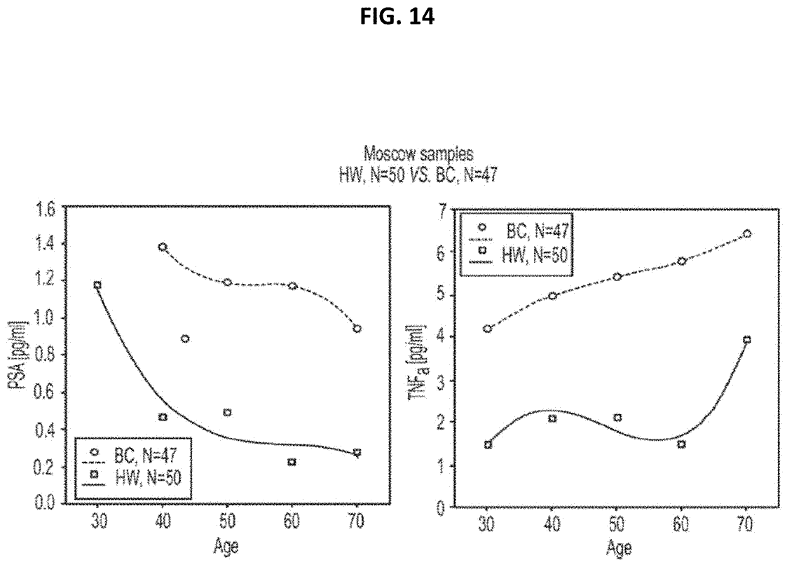

FIG. 14 shows such functions for breast cancer and not-breast cancer from market clearance trials conducted at the Gertsen Institute Moscow for TNFα and Kallikrein 3 (PSA). Note that this plot can give very good indications of the biomarker that will yield predictive power when coupled with other biomarkers in the manner described in this application. The degree of separation, across all ages indicates, from the measurement science perspective, that there is a strong “signal” that will differentiate from the not signal condition, disease and not-disease will differentiate. In most cases, this will give a better indication of predictive power than a single ROC curve.

### Use of Functionally Orthogonal Biomarkers and the Spatial Proximity Correlation Methods

The method uses the Spatial Proximity search (neighborhood search) for correlation. This method places each independent variable on a spatial axis, and each biomarker used has its own axis. Five biomarkers are placed in a 5 dimensional space. Each biomarker is transformed by the meta-variable method as discussed herein. This method forces the normalization of age related drift in concentration actions and immune system non-linearity. The test panel discussed here is for breast cancer and it uses an inflammatory marker, Interleukin 6; tumor anti-angiogenesis or cell apoptosis marker, Tumor Necrosis Factor alpha; and tumor vascularization markers, Vascular endothelial growth factor (VEGF); and an angiogenesis marker, Interleukin 8; as well as a known tumor tissue marker, kallikrein-3 (or PSA). These markers are highly complementary in the proximity method for correlation as their functions do not overlap significantly. Thus, when plotted orthogonally, they enhance separation as each added axis pulls the biomarker data points apart, for not-cancer and cancer as shown in the Figures. Other standard correlation methods such as regression analysis or ROC curve area maximization methods cannot retain this orthogonal separation as the mathematics analysis looks for individual marker trends (linear regression—linear and logistic—logarithmic). Any spatial information is lost.

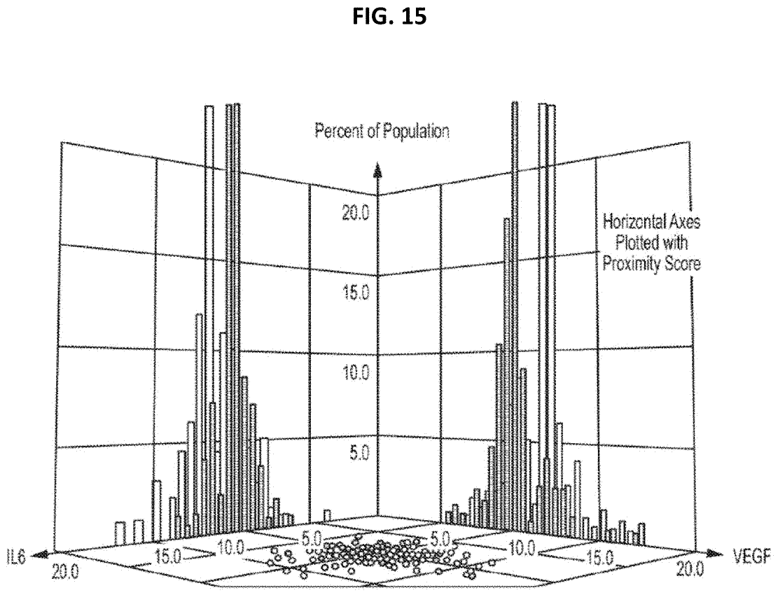

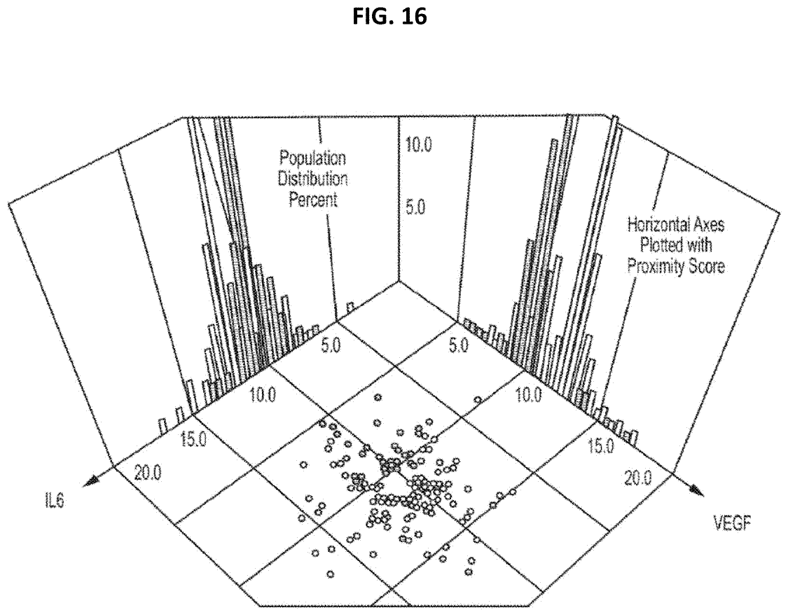

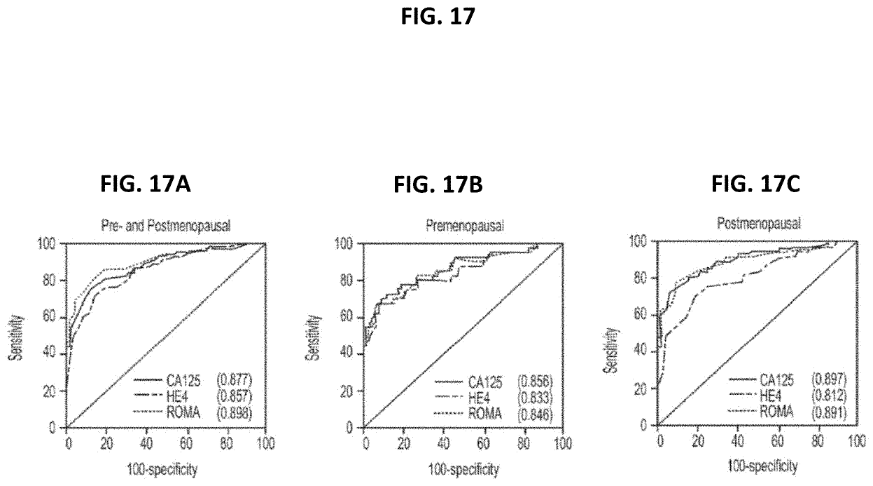

The phenomena noted above, orthogonality or incongruence of function, can also be seen graphically in FIGS. 15 and 16. These graphs show the concentration population distribution of the pro-inflammatory biomarker, IL 6 plotted against the vascularization biomarker VEGF on the horizontal orthogonal axes. FIG. 15 shows the 3D plot rotated so the horizontal plane is nearly horizontal, and FIG. 16 shows this x, y plane rotated so the planar distribution of the markers can be seen on this horizontal plane. The horizontal concentration axes show this parameter plotted not in concentration units but the in the Proximity Score computed as discussed herein. The vertical axis shows population distribution as a percentage of the total. The bin size is 0.5 units of Proximity Score for each vertical bar. Note that this graphic plotting depiction will not allow side by side separation of the two population groups, not-cancer (bl and cancer. Thus, the bars overlay each other. When the not-cancer population is higher than the cancer population, the cancer population shows above the cancer population and vice versa, but they do not add, the cancer population behind the not-cancer population still shows the cancer population high as correct on the vertical axis. Note the considerable overlap of the not-cancer on the cancer population and vice versa, as one would expect with any one biomarker. Also note that the cancer populations are generally at higher Proximity Score levels along each axis compared to the not-cancer samples, as one would expect with a single biomarker. FIG. 6 shows these same 3D axes rotated 45° down to show the horizontal axes. Note the dramatic separation of the individual markers. The pro-inflammatory markers, IL 6, that show a low response, but are cancer, tend to show a high level vascularization response, and vice versa. This effect would be expected by any biomarker chosen for its uncoupled functionality with respect to the other biomarkers chosen and where the biomarkers up regulate in general to the cancer. This would be expected by simple probability, both proteins up regulate in the disease transition, and those with a low response from one function will likely show a stronger response from the other. This effect is even more enhanced in breast cancer with the orthogonality of the inflammatory and vascularization functions. FIGS. 17A-C show the degree of up regulation of each of these proteins in breast cancer by cancer stage. Note that the pro-inflammatory marker up regulates highly first at the onset of the nascent stage 0. However, as the tumor progresses, the vascularization marker up regulates to a greater degree as the tumor grows, stage 1 through 4. Thus, low level pro-inflammatory response, late stage, is coupled with high level vascularization response. And high level pro-inflammatory response is coupled with relatively low level vascularization response in the early stage of the disease. This behavior, when plotted in a multi-dimensional correlation method, will separate, in cancer, low level vascularization response with high level pro-inflammatory response, pulling these sample points away from the origin (and vice versa for the opposite). The correlation information is in the pull by function away from the orthogonal axis for the other function, in cancer. Note that this enhancement is lost in methods such as regression or ROC curve area maximization as the coupling of the orthogonal functions is lost.

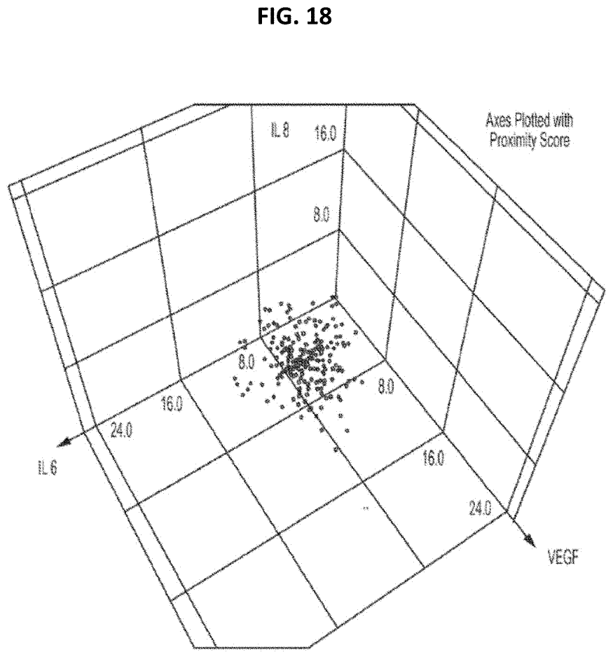

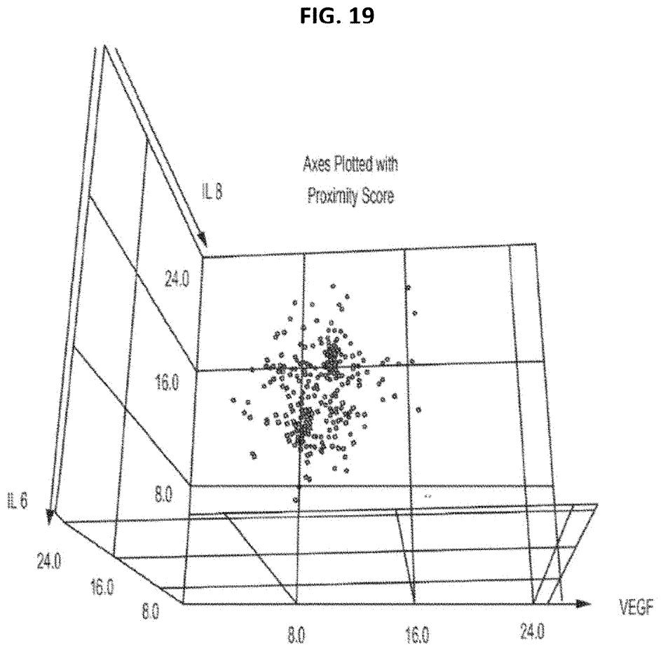

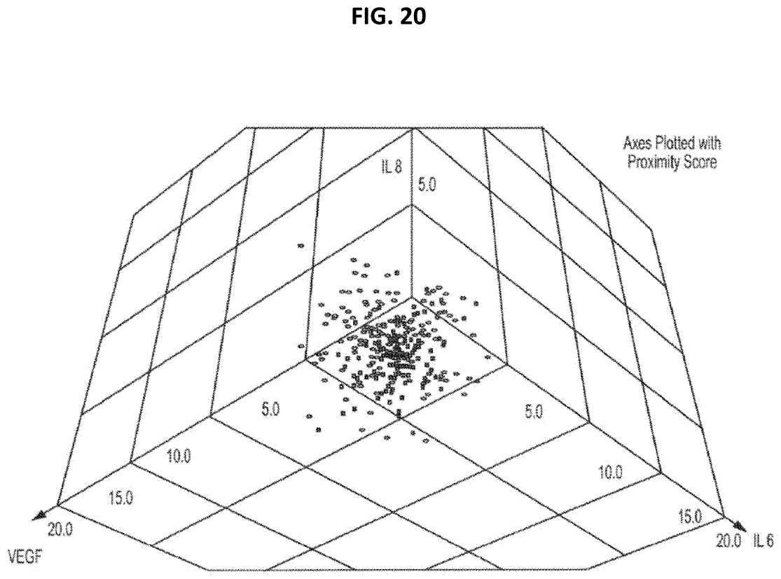

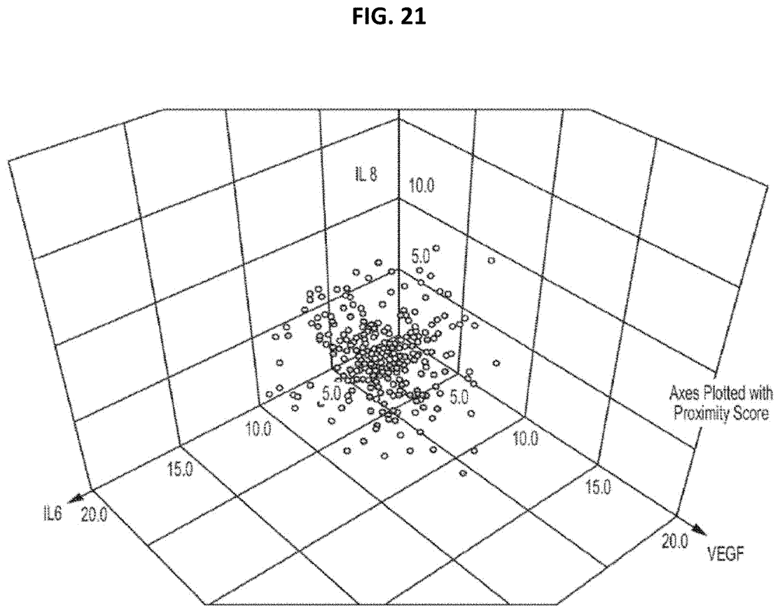

FIGS. 18 through 21 show a third biomarker IL 8, primarily an angiogenesis function in 3D with the other two discussed above. Note that angiogenesis, IL 8, and vascularization, VEGF, are both involved in growing blood vessels but are not the same. Angiogenesis, IL 8, drives creation of blood vessels from tissues with existing circulation and vascularization, VEGF, drives production of new blood vessels in bulk tissue where there are no pre-existing ones. Tumors are known to produce both responses. Again, looking at FIG. 17, angiogenesis is strong in the early stage when the tumor is within vascularized tissue and vascularization increases as the bulk tumor grows. The plots are: FIG. 18 shows the plot looking down into the plot origin at 45° from above for all axes. FIG. 19 shows the plot rotated showing the horizontal axes ten degrees above horizontal and the vertical axis rotated about 35° to the right. The not-cancer are clearly located below the cancer and closer to the origin. FIG. 20 shows the whole plot rotated around to the back side to look through the origin to the not-cancer with the cancer in back, FIG. 21 shows the plot rotated up slightly to show the cancer in front of the not-cancer. Note that this separation is greatly enhanced by not using actual concentration but the Proximity Score discussed in related applications, as outlined above and in this application. These plots clearly show how selecting biomarkers with complimentary functions, (i.e., orthogonal) yield significant improvements in separation and thus predictive power. This improvement will continue through the other two markers not shown, TNFα (anti-tumor genesis), and Kallikrein 3 (PSA) tumor marker. They can't be plotted with the first three, of course, as this would exceed 3 dimensions, and the eye cannot see this. These two markers, when plotted against one of the three noted above, will look substantially the same, showing a high degree of separation on each axis. The computerized 5-dimensional Spatial Proximity correlation method retains this orthogonality.

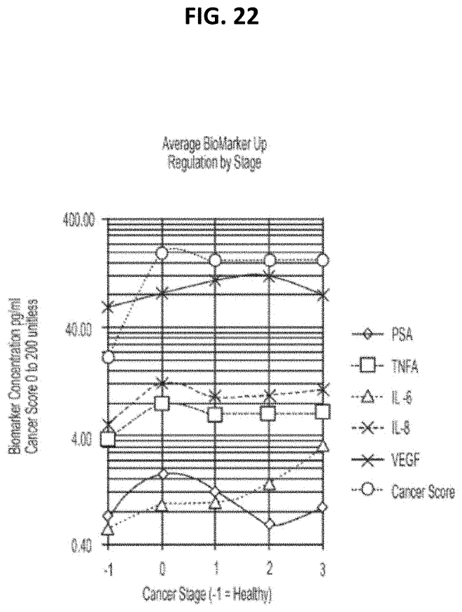

In summary, the nascent breast cancer tumor, stage 0, develops a very strong pro-inflammatory response, as shown in FIG. 22. This response by itself cannot be differentiated from infections, allergies or autoimmune disease (and others). However, this same nascent tumor will generate a strong angiogenesis response, circulatory increases in vascularized surrounding tissue. Thus, in FIGS. 18 through 21, the nascent tumor samples will move out on the pro-inflammatory axes and up the angiogenesis axis (and the tumor anti-genesis axis and tumor biomarker axis in the fourth and fifth dimensions). A late stage tumor stage 3 or 4 will tend to show a strong vascularization response (growth in bulk tumor tissue without vascularization) and a weaker anti-tumor genesis, moving out from the origin on the VEGF axis. These cannot be discriminated from trauma wounds, cardiac ischemia or pregnancy as these conditions call for vascularization. However, again, unrelated functions, tumor anti-genesis and up regulation of the tumor marker will create the differentiation.

This improvement is multiplied as the other three biomarkers are added to the 5-dimensional correlation grid. This careful selection of biomarkers for incongruent functionality improves predictive power over methods where multiple tumor markers are selected. Tumor markers for the same tumor tend to measure the same phenomena and this will not pull the biomarkers apart on these orthogonal axes and they will just rotate the group clustering by 45 degrees. Regression and other methods do not retain this orthogonal information. This improvement can only be achieved with functionally orthogonal biomarkers and the Spatial Proximity correlation method.

The measured concentration values themselves are not used in the 5 axis grid for the Spatial Proximity correlation. The Proximity Score is used. This computed value removes age related drifts in the transition from not-cancer to cancer, the age variation in the mean value of actual concentration, not-cancer and cancer are normalized. Also, actual concentration is carefully expanded and compressed to eliminate what we call local spatial and population density biases to determine the value of the Proximity Score. This number is unit less and varies over an arbitrary range of 0 to 20. These two corrections will improve predictive power by about 6%. The use of incongruent functional cytokine groups will achieve about 10% to 15% higher predictive power than using multiple tumor markers as biomarkers. The normalization of age drift and non-linear up down regulation produces a 6 to 7% improvement in predictive power over conventional proximity search methods.

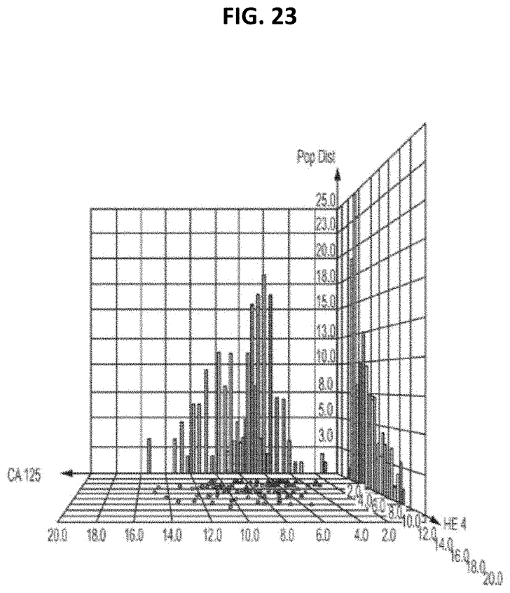

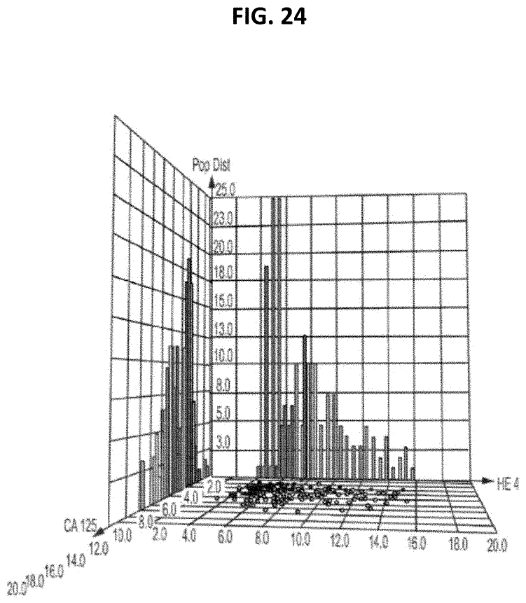

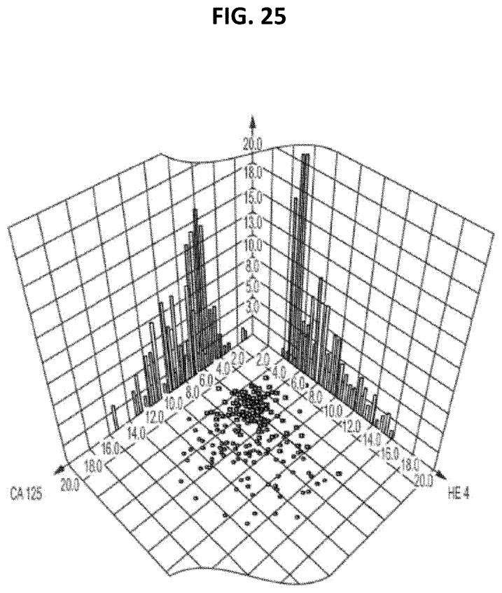

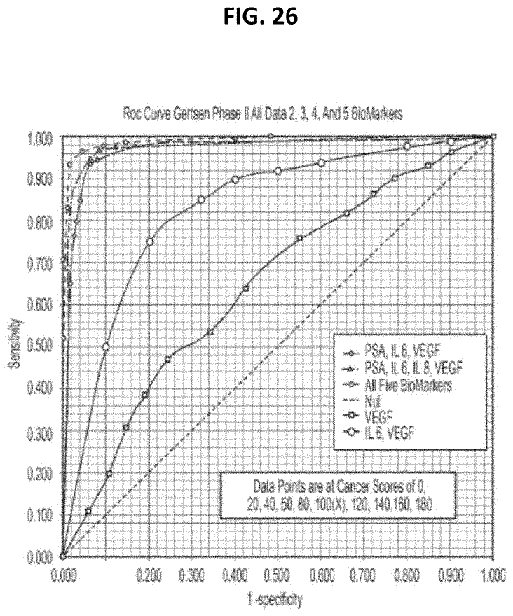

In contrast, FIGS. 23, 24, and 25 show population distribution of CA 125, HE4 for ovarian cancer, again on the horizontal axes and population distribution on the vertical axis. FIG. 13 shows these axes rotated down to see the orthogonal relationship of these biomarkers to each other. This 3D plot also shows the spatial distribution of these two markers when plotted on the horizontal 2-dimensional bi-marker plane (the vertical axis shows population distribution). The concentration is plotted as the normalized log concentration ranged from 1 to 20. CA 125 and HE4 are well known ovarian cancer biomarkers. In fact, for single high abundance protein cancer markers, these are very good. HE4 is far better than PSA for prostate cancer in men. Yet they are not good enough for regulatory approval for screening. Even the combination of the two is not effective. Note that the single biomarker is relatively good for both. CA 125 will achieve about 50% specificity at 90% sensitivity. HE4 will achieve about 45% specificity at 90% sensitivity. Notice that the orthogonal separation is not much different when viewed in two dimensions than for the single biomarker by itself “HE4 a novel tumor marker for ovarian cancer: comparison with CA 125 and ROMA algorithm in patients with gynaecological diseases;” Rafael Molina, Jose M. Escudero, Jose M. Augé, Xavier Filella, Laura Foj, Aureli Torné, Jose Lejarcegui, Jaume Pahisa; Tumor Biology; December 2011, Volume 32, Issue 6, pp 1087-1095. FIG. 15 shows the addition of AFP, another general and ovarian cancer biomarker. No additional improvement is seen over CA 125 and HE4. These three biomarkers are measuring similar aspects of the same thing and thus are not complimentary in improving predictive power when viewed with orthogonality maintained. The combined performance (using standard methods) is about the same as HE4 by itself. FIG. 16 shows the ROC curves for CA125 and HE4 alone and then the combined ROC curve for the two when correlated to ovarian cancer. The combination is nearly an overlay of the HE4 ROC curve. There is no improvement in performance at all (except a slight improvement for post-menopausal women). “HE4 and CA 125 as a diagnostic test in ovarian cancer: prospective validation of the Risk of Ovarian Malignancy Algorithm;” T Van Gorp, I Cadron, E Despierre, A Leunen, F Amant, D Timmerman, B De Moor, I Vergote; Br J Cancer, Mar. 1, 2011; 104 (5) 863-870. The dramatic improvement in ROC curve using three, then four, and then all five biomarkers with this so-called orthogonal function characteristic, is shown in FIG. 26. These plots all use the logarithm of the raw concentration. Note that if these raw concentrations were converted to Proximity Scores, an improvement would be seen as the orthogonal separation movement is enhanced when the Proteomic variance “noise” is removed. Shear probabilities indicate that a tumor biomarker for one cancer with a low response will likely have a higher response on an orthogonal axis, when this noise is suppressed.

Further separation occurs on this orthogonal grid by just the conversion to Proximity Score. FIGS. 15 and 16 show the data in FIG. 10 on the 3D plot where the vertical axis is the population distribution of each biomarker. The Proximity Score separates the sample data into two groups, populated by, mostly not-breast cancer close to the origin and breast cancer far away from the origin. These distributions are approximately Poisson. Notice the normal single biomarker overlap on each of the horizontal axes. No amount of mathematical manipulation can get rid of this problem. Notice however, that individual Breast Cancer samples that are low on the pro-inflammatory axis (IL 6) tend to have a high position on the vascularization (VEGF) axis. The same is true of the other horizontal axis for (VEGF). Note that this separation will occur where functionally orthogonal biomarkers are used, or with tumor markers that do not have inherent orthogonal separation actions. Simple odds will dictate that a low level concentration for one of the tumor markers will very likely correspond with high levels for all the others in a cancer patient. For example, if a test panel includes 5 tumor markers (not orthogonal in action), the markers are measuring the same condition (e.g., a tumor is present). All the markers up regulate for the most part. If one marker has a poor response, for example is not present at levels typically found when up regulated, in an individual, it is likely that the others must also be active up regulating as well. This separation action is brought out when the Proteomic Variance (or noise) is dampened. Within the raw concentration values, this separation effect is contaminated by the noise. Note also that this separation keeps piling up through all, in this example, 5 orthogonal dimensions in the grid, whether the biomarkers are chosen for orthogonality of function or are just tumor makers that indicate the presence of the same tumor, with the orthogonality of function having by far the best separation. Note that each of these dimensions are associated with each biomarker selected. Thus, five biomarkers will require 5 dimensions, and 6 biomarkers requires 6 dimensions, etc.

### Spatial Proximity Method

The methods include a multi-dimensional space, one for each biomarker. The Proximity Score for each biomarker in the Training Set is plotted in the multi-dimensional space (5 dimensions in this breast cancer example). The plot is broken up into a grid, and then each point in this five-dimensional grid is scored breast cancer or not-breast cancer by its closest proximity to several (5 to 15 percent) Training Set points on the grid. The cancer score is rendered by the count of breast cancer and not-breast cancer in the local vicinity of the empty grid point being scored. Maximum score is achieved in the empty grid point when it “sees” only breast cancer and vice-versa for not-breast cancer. Unknown samples are then placed on this grid and scored accordingly. Table 1 shows that combining this functional orthogonal selection of biomarkers with the Proximity Score Conversion (noise reduction and age normalization) yields predictive power of 96% for these biomarkers in this breast cancer case.

TABLE 1

| | Correlation | Predictive | Improvement |
|---|---|---|---|
| Data Manipulation Method | Method | Power | Over Baseline |
| Logarithm of Raw concentration | Logistic Regression | 80% | Baseline |
| Logarithm of Raw concentration | Neural Network | 84% | 4% |
| Logarithm of Raw concentration | Surface Vector | 84% | 4% |
| | Machine | | |
| Conversion of Concentration to | Logistic Regression | 85% | 5% |
| Proximity Score | | | |
| Conversion of Concentration to | Neural Network | 87% | 7% |
| Proximity Score | | | |
| Conversion of Concentration to | Surface Vector | 90% | 10% |
| Proximity Score | Machine | | |
| Conversion of Concentration to | Spatial Proximity | 90% | 10% |
| Proximity Score | | | |
| Conversion of Concentration to | Spatial Proximity | 96% | 12% |
| Proximity Score plus Orthogonal | | | |
| Biomarkers | | | |
| Plus Correction of Blind Samples | Spatial Proximity | 96% Plus | 12% plus |
| for Topology Instability | | | |

This can also be done on individual bi-marker slices through the 5-dimensional grid on each biomarker two-dimensional plane to reduce computation time. This produces 10 so-called bi-marker planes. The 2-dimensional grid point is again scored by proximity to the training sets, disease or not-disease by the 2-dimensional proximity to the training set points. In this case, 3 to 10 percent of the closest data points are used for the proximity distance. This yields scores for each grid point. Grid points with a training set data point in it ignore the actual diagnosis of that training set point for the grid point score. The plane is then scored for predictive power, sensitivity and specificity by counting the training set points correct versus not correct by the usual definitions. The 10 resulting planes are then added up with an individual plane predictive power weighting. This weighting of each bi-marker plane is the predictive power (also sensitivity can be used) of that plane. The additive score of all ten planes is then shifted and gained to get a range from 0 to 200 with 0 to 100 labeled as not-cancer and 101 to 200 labeled as cancer. Unknown sample data points are then scored by their placement on these bi-markers planes by the predetermined scoring from the model build using the training sets.

### ROC Curves for a Five-Biomarker Breast Cancer Diagnostic Test Panel

FIG. 26 shows the combined ROC curves for the full 5 test panel derived from the concentration values measured at the Gertsen Institute for cancer and not-cancer cohorts of 407 serum samples total. This overall plot shows five ROC curves: 1) VEGF alone; 2) IL 6 and VEGF combined; 3) PSA, IL 6 and VEGF only; 4) PSA, IL 6, VEGF and IL 8 only; and 5) all five biomarkers. The buildup of predictive power is clear when looking at the cancer score set points corresponding to 100, the mid-point between the arbitrary 0 to 200 cancer score range. FIG. 18 shows this range of the ROC curve blown up to better see the improvement achieved with each added biomarker. The X mark is on the data point for the midpoint cancer score of 100. This would be the putative transition point from not-cancer to cancer. Though medical goals may shift this value. Oncologists have set the transition point at about 80 to minimize false negative predictions at the expense of false positives results. These data show all data set points, both the training set and the blind samples as well as data from a third party validation of the OTraces BC Sera Dx test kit for detecting breast cancer, for a total of 407 data sets. Note that the predictive power within the training set and the final predictive power scoring of the blind data set had about the same predictive power, about 97% to 98%. The reported cancer score in this case is an arbitrary scoring from 0 to 200 with 0 to 100 being not-cancer and 100 to 200 being cancer. Note that the curve for all five biomarkers does not terminate at the usual axis end points, 0,0 and 1, 1. This is because a significant number of the data set points have a cancer score of exactly 0 and 200. 30% of the not-cancer samples have a score of 0 and about 50% of the cancer points have a score of 200. These points in the 5-dimensional grid only see respectively not-cancer for the 0 scores and cancer for the 200 score of the training set points in the grid. The proximity test uses the three closest points for the score computation on each 2-dimensional orthogonal cuts through the 5-dimensional space. These cuts are called bi-marker planes. The 5-dimensional space yields 10 discrete bi-marker planes. In the full five dimensions each blind sample is tested for proximity to about 20 to 25 different training set data points. These samples that score 0 or 200 see only not-cancer or cancer training set points, respectively in the grid. Thus, they score respectively 0 and 200, the ends of the arbitrary range. The same is true, but to a lesser extent for the 3 and 4 biomarker curves. This demonstrates the robustness of the method.

Though these biomarkers have insufficient predictive power to be used as a screening test, combined they can achieve predictive power above 95%. However, this performance cannot be determined from individual ROC curves and the measurements of one biomarker's behavior. VEGF has the poorest performing ROC curve but when combined with the pro-inflammatory biomarker shows a very high boost in predictive power. This is due to amplifying effect of the orthogonal functions of these biomarkers. Furthermore, biomarkers with these features continue to amplify predictive power. This amplification can only be seen when the orthogonal information contained within the multiple functions is retained in the Spatial Proximity correlation method.

Assessing the performance of one biomarker by itself has limited value. They need to be assessed in a multi-dimensional format where coupling (or uncoupling) of functionality is maintained. Alternately, the biomarkers can be studied in an orthogonal matrix. This amplification of predictive power shown in these ROC curves comes directly from: 1) the suppression of Proteomics Variance by conversion to Proximity Score; 2) the use of biomarkers with Functional Orthogonality coupled with the Spatial Proximity correlation method; and 3) Normalization of the age drift inherent to the transition from not-disease to disease.

### Age Normalization

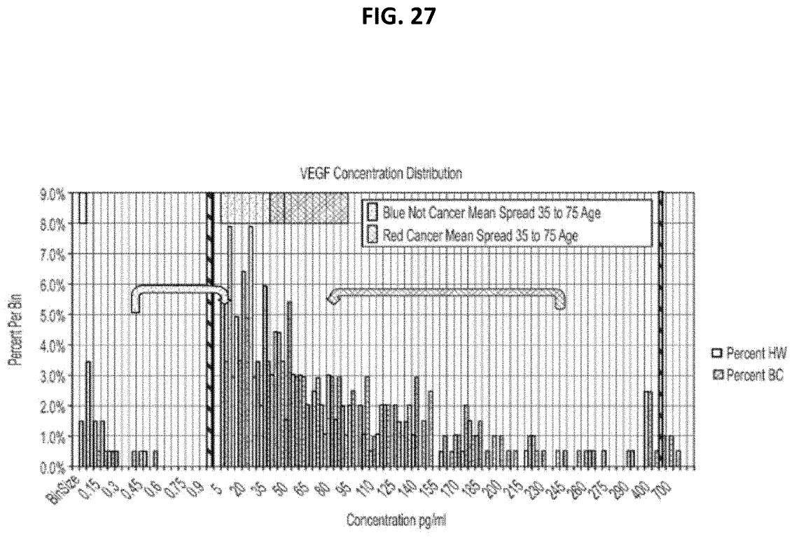

The measured concentration distribution of VEGF in female humans is measured in about 400 patients in FIG. 27. VEGF is an anti-tumor low abundance cytokine that is up-regulated generally in serum with the presence of cancer but also up-regulates in other conditions.

Age causes a complication to the above discussion as the population mean values for both not-cancer and cancer change with age. Additionally, using age as a separate independent variable in the correlation analysis does not improve predictive power. Thus, though the methods described above improve predictive power, age drift should be factored into it. Related provisional application 61/851,867 (and its progeny) describes how to use age as a meta-variable in the transformation of the concentration variables into age factored Proximity Score values. The discussion below describes methods to improve this transformation.

As outlined previously, methods for improving disease prediction can use an independent variable for the correlation analysis that is not the concentration of the measured analytes directly but a calculated value (Proximity Score) that is computed from the concentration but is also normalized for certain age (or other physiological parameters) to remove such parameter's negative characteristics such as age drift and non-linearities in how the concentration values drift or shift with the physiological parameter (age) as the disease state shifts from healthy to disease. This discussion provides improvements to that method.

One equation for conversion of concentration to Proximity Score discussed in the application is (see possible equations for the concentration to Proximity Score Conversion above, and also reproduced below):

PSₕ=K*logarithm₁₀((Ci/C₍ₕ))−(Cc/Ch))+Offset Equation 1

PS_{c}=K*logarithm₁₀((Ci/Cc)−(Ch/Cc))²+Offset Equation 2

Where:

PSh=Proximity Score for not-cancer

PSc=Proximity Score for cancer

K=gain factor to set arbitrary range

Ci=measured concentration of the actual patient's analyte

Ch=patient age adjusted mean concentration of non-disease patients' analyte

Cc=patient age adjusted mean concentration of disease patients' analyte.

Offset=Ordinate offset to set numerical range (arbitrary)

This is referred to as equation 1 and 2 in the text below.

These equations selectively compress or expand measured concentration values to allow a better fit to the proximity correlation method. Age adjusted mean concentration values are used for the not-disease state and for the disease state. The method for age adjustment below shows that this improved method uses this equation and others in portions or zones on the graph showing the measured concentration and resultant Proximity Score that is actually used in the correlation analysis.

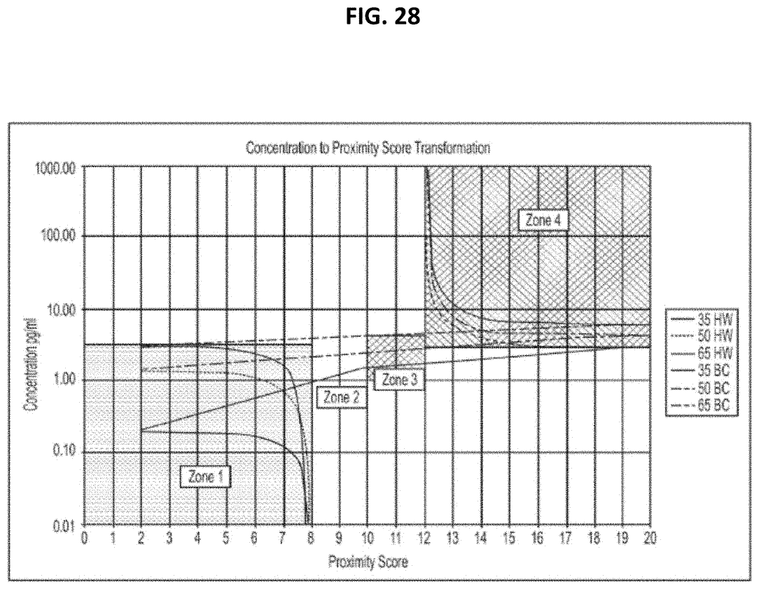

FIG. 28 shows Equation 1 and Equation 2 plotted showing the conversion from concentration to Proximity Score. Note that Equation 2 is inverted and reversed mathematically and its offset value is shifted such that the not-cancer equation (one) does not overlap the cancer equation (two) on the ordinate. The age related mean values are shown on the abscissa as the horizontal asymptotic curves not-cancer going to the left and cancer going to the right. These asymptotic curves vary with age again on the abscissa. In fact, for some markers, the age adjusted mean value for not-cancer and cancer overlap on the vertical axis, as shown on the figure. This aspect of the biology of this particularly deteriorates the predictive power if not dealt with. This embodiment shows Zone 1 folds onto Zone 2 and Zone 4 folded back on Zone 3 (see discussion on Population Distribution Bias). In the case of cancer versus not-cancer the cancer cohort is over represented in the training set by a large margin. The folding improves the distribution bias in the zones dominated by not-cancer.

FIG. 13 shows an alternate embodiment that uses a straight log concentration to linear conversion. In this scenario, PS=M(log(Ci)+B, where PS=Proximity Score (the concentration), Ci=the measured concentration of the actual patient's analyte, M=the conversion slope, and B=the offset. Again, this embodiment shows Zone 1 folds onto Zone 2 and Zone 4 folded back on Zone 3.

The equations and resulting Proximity Score values are forced into zones on the two-dimensional plot by adjusting the offset values. Furthermore, all individual samples at a particular age with actual measured values below that age mean values for not-cancer will be forced into zone 1. Likewise, all samples at a particular age with actual measured values above the mean value for cancer at that age are forced into zone 4. Similarly, samples with actual values between the mean value of not-cancer at that age at particular age and the midpoint between not-cancer and cancer mean values for that age are forced into zone 2, likewise for zone 3. In effect, the Proximity Score forces the individual sample of a certain age to take one of four positions based upon its relationship to the mean values for not-cancer and cancer for that age. The Proximity Score forces the concentration measurement to take sides. Note that this does not indicate that say a sample in zone 1 will be not-cancer. That depends on how the other four markers behave. The three key points not-cancer mean, cancer mean, and the derived midpoint between them, all vary independently on the abscissa and may overlap but are normalized in set zones or values on the ordinate (Proximity Score).

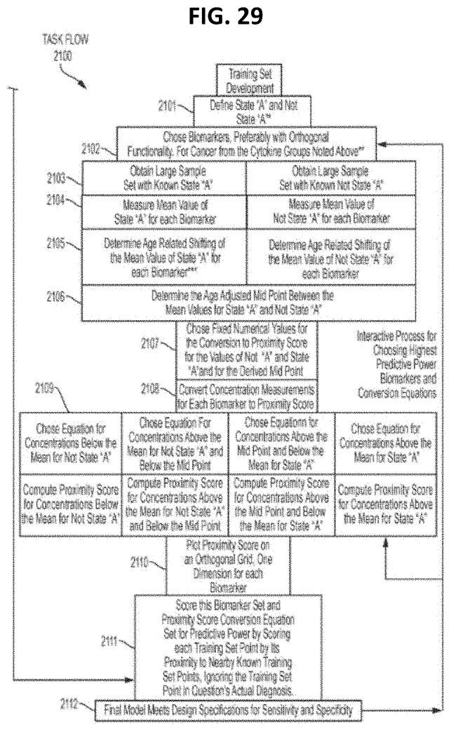

FIG. 29 depicts an exemplary flow chart for Building Proteomic Noise Suppression Correlation Method. This flow chart describes the steps involved in developing a high performance correlation algorithm for separating two opposing conditions (state “A” and not-state “A”) needed for diagnosis of either a disease state, a condition within a disease state related to severity or to determine the best population suitable for treatment of the disease with a particular drug. State “A” and Not-State “A” could be the presence of a disease and absence of the disease. Alternatively, it could be a severe state of the disease and a less severe state of the disease. Also, it could be for scoring a particular drug or treatment modality for efficacy within a group of prospective patients. For cancer, the preferred cytokines with orthogonal functionality would be: pro-inflammatory, anti-inflammatory, Anti-tumor genesis, angiogenesis, and vascularization. Also, at least one tumor marker would be appropriate. Age could a different independent variable. We term this variable the meta-variable. In addition, it should be noted that age Body Mass index, race, and geographical territory, among other independent variables, are possible as meta-variables.

An exemplary method is shown in as 2100, “Task Flow.” At step 2101, State “A”, exemplarily the Disease State, and Not-State “A”, exemplarily the Non-Disease State, are defined. At step 2102, biomarkers comprising the set are chosen, preferably those with orthogonal functionality. At step 2103, large sample sets of known State “A” and Not-State “A” are obtained. At step 2104, for State “A” and Not-State “A,” the mean value for each biomarker is measured. At step 2105, for State “A” and Not-State “A,” age-related shifting is calculated. At step 2106, the age-adjusted midpoint between the mean values for State “A” and Not-State “A” is calculated. At step 2107, the software calculates fixed numerical values for the conversion to Proximity Score for the mean values of Not-State “A” and State “A” and for the derived midpoint. At step 2108, the concentration measurements for each biomarker in the set are converted to a Proximity Score. At step 2109, the biomarker Proximity Scores for each biomarker in the set are used to compute concentration Proximity Scores and choose equations for concentration for State “A” and Not-State “A”. At step 2110, the Proximity Score is plotted on an orthogonal grid, such that there is one dimension for each biomarker in the set. At step 2111, the biomarker set is scored, based on, for example, the Proximity Score Conversion Equation Set. This biomarker set score results in the highly predictive method for diagnosis discussed herein.

### Negative Aspects of the Spatial Proximity Correlation Method

The Spatial Proximity Correlation method has very significant advantages over other methods in that it retains the orthogonal spatial separation inherent in these biomarkers as the transition from healthy to cancer occurs. However, the method may have several disadvantages that are not relevant to conventional analytical approaches that can be overcome. The method plots the training set data on a multidimensional grid and then scores other “blind” (not occupied) points on the grid for not-cancer or cancer by proximity to the training set points. The best correlation performance generally occurs if the movement of these biomarker data points is relatively linear. That is, if the movement or up/down regulation is highly non-linear or exhibits clumping with highly isolated points, degradation of the correlation may occur. Basically, highly isolated points on the grid will influence all nearby points with the scoring of the isolated point at the expense of others. A second problem is related to the relative general population distribution of the training set data and the real distribution of the disease in the general population. In the case of breast cancer, the general population distribution is about 0.5% cancer to 99.5% not-cancer. Yet the training set must be distributed 50%/50% or it will bias the correlation in favor of the side with higher population. No bias demands the 50%/50% split. This may cause areas with predominant not-cancer but low levels of cancer to over call cancer in these areas and vice versa.

### Special Bias Problems With the Spatial Proximity Correlation Method and Human Biological Measurements

FIG. 27 shows the population distribution of one of the biomarkers discussed for the cancer predictive test. This non-linear distribution with clumping and highly isolated data points is typical for all five of these biomarkers and most, if not all, of these low level signaling proteins (cytokines). This is indicative of the non-linear behavior of the immune system. This problem (and the age shift effect described above) significantly decays the ability to correlate these proteins to disease state predictions. This example is intended to teach how to correct this non-linear up regulation behavior.

In FIG. 27, the concentration distribution is highly non-linear with blocks of concentration values at extremely low levels as well as very high levels. This is an indication of the non-linear behavior of the immune system. This behavior is common to all of these cytokine or signaling based biomarkers. In fact, the biomarkers used in this breast cancer detection method discussed herein all look very similar to the plot in FIG. 27. Also note that the distribution shows isolated points in between the clumps. This will cause a correlation bias we term “Local Spatial Distribution Bias.” Both of those deficiencies are partially mitigated with the use of Equations 1 and 2, as disclosed above.

### Local Spatial Distribution Bias

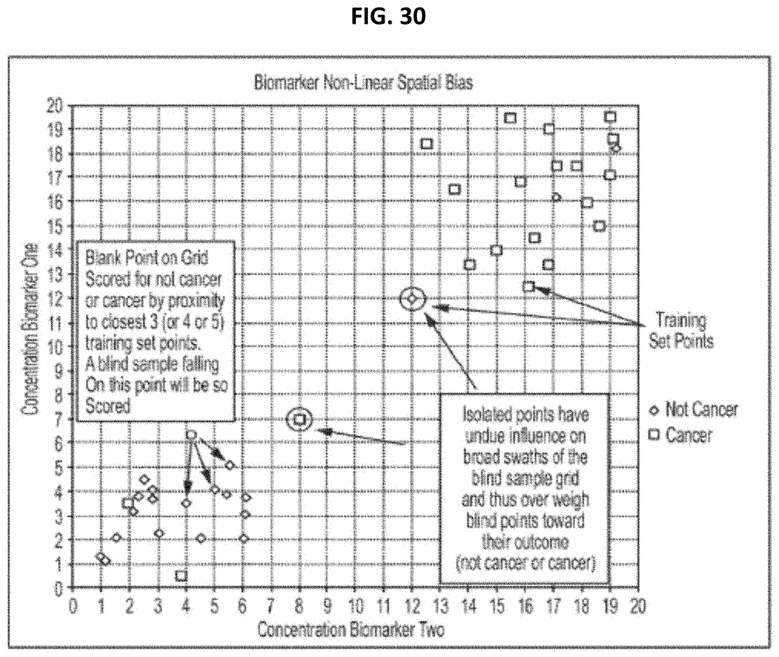

As noted above, this problem is partially mitigated by the use of Equations 1 and 2, though there may be many other possible solutions. FIG. 30 shows a stylized two dimensional biomarker plot showing cancer at high levels and dispersed. Also, not-cancer is shown at lower levels and compacted. Isolated points between these clumps are also shown. The standard deviation of the spacing of the plot points on this graph is about 8 units. Note that the two isolated points on the graph will sweep up large sections of the proximity plot forcing these areas with the isolated point's diagnosis.

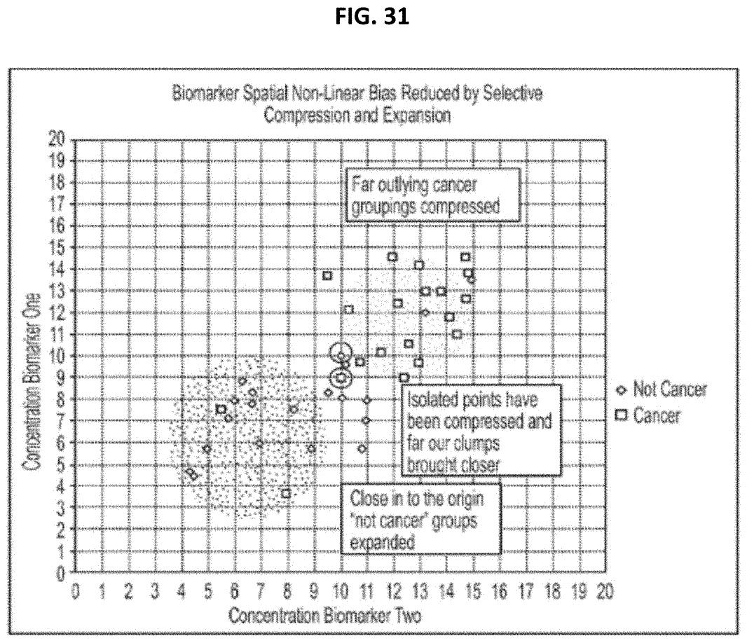

FIG. 31 shows these same points conditioned by the compression and expansion performed by Equations 1 and 2. The standard deviation between points on this graph is about 2.5 and the clustering and isolation are very much reduced. This mathematical manipulation is perfectly acceptable under the rules noted above under the discussion of the measurement science. Indeed, the distance standard deviation reduction is a good rule of thumb for predictive power of the model. Note the standard deviation of the spacing is reduced to only 3 units. This spacing deviation should be as low as possible without shifting the spacing order.

### Population Distribution Local Bias

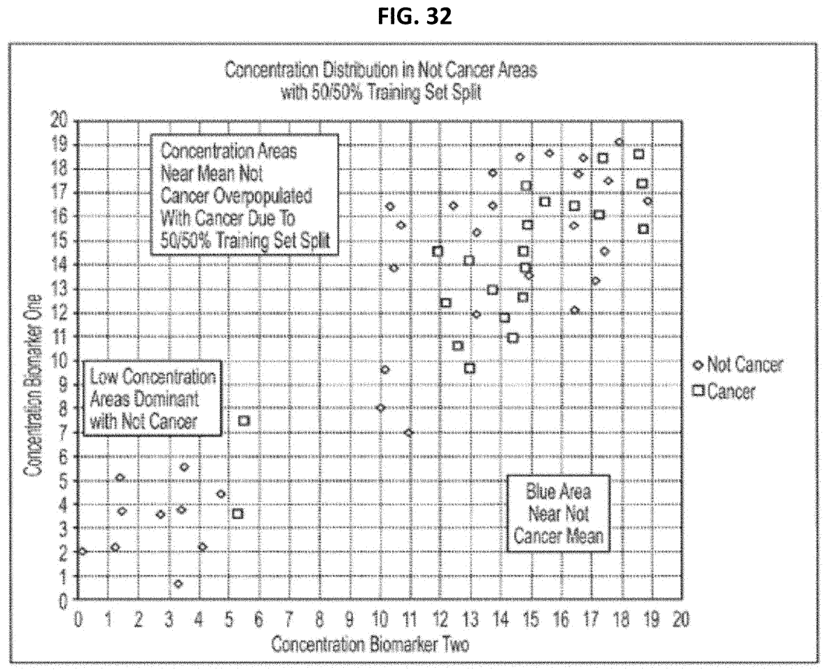

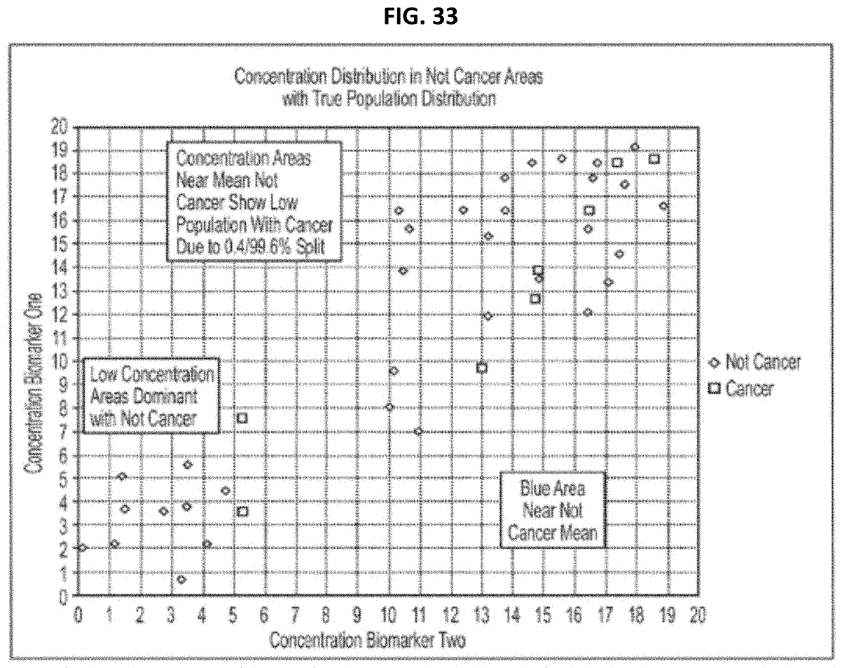

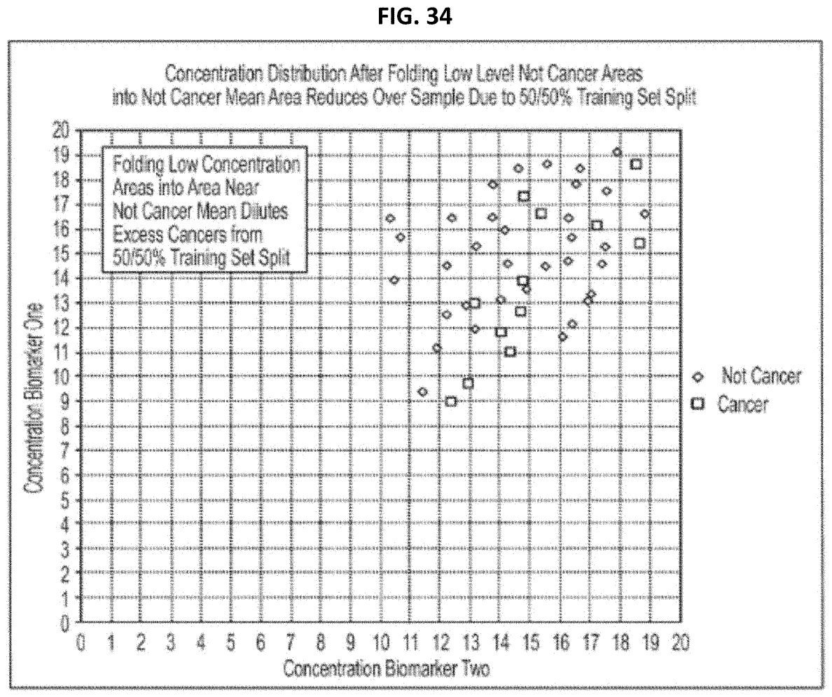

FIGS. 32, 33, and 34 show how this issue can be mitigated. FIG. 32 shows the over representation of cancer in the not-cancer space for samples below the age related mean value for not-cancer. The area in the upper right will generally be over samples with cancer. The samples in the lower left are dominated by not-cancer and thus are more correct. FIG. 33 shows how the plot would look if properly represented by the real lesser distribution of cancer. These are at risk of bias and can be mitigated to a degree by folding the lower right area up into the areas near the age related mean value for not-cancer. These very low concentration values, well below 1 pg/ml, are populated into the higher concentration area, helping mitigate the bias. The stylized plot showing the folding and reduced local population distribution bias is shown in FIG. 34.

The mathematical rules are: 1) The training set model should be populated by 50% not-cancer and 50% cancer to remove model bias. 2) Mathematical manipulations are acceptable for reducing the effect of the physical characteristics of the independent measurement to reduce the effect of extraneous informant noise provided the methods are applied to both the training set model and the blind samples to be tested.

Using simple logistic regression with these biomarkers for breast cancer will yield predicative power of slightly less than 80%. Using simple standard Spatial Proximity correlation without the age and non-linearity corrections (simple logarithm of concentration) yields about 89% predictive power. These improvements discussed above: 1) age normalization; 2) local spatial distribution bias corrections; and 3) population distribution local bias corrections, yields about 96% predictive power with these biomarkers. Adding correction of blind samples for topology instability can add another 1 to 2% improvement.

### Spatial Bias and Population Distribution Bias Corrections are Complementary to the Variance (Noise) Suppression Methods

The methods discussed above for correcting two bias problems associated with the Spatial Proximity Correlation method are complimentary to solving the problem of Proteomics variance (noise). The correction methods both involve compressing the raw concentration data, and this compression is toward the predetermined mean values for disease and not-disease. In fact, correcting the population bias problem involves folding the very low concentration values (well below the not-disease mean) into an area near or even above the not-disease mean. The same is true of the very high concentration values.

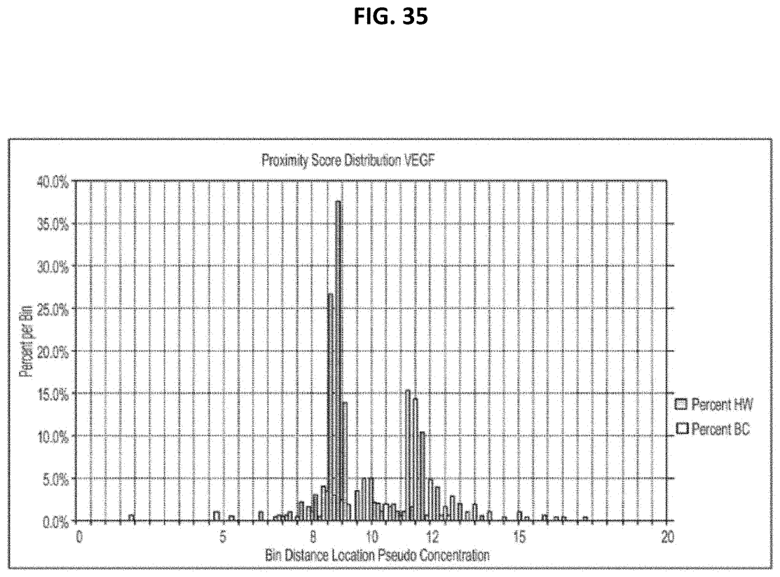

The resulting Proximity Score distribution of this method is shown in FIG. 35 for VEGF. The other four look similar. The process forces sample data points into two roughly overlapping Poisson distributions where not-cancer predominates on the lower side and cancer predominates on the upper side. Note that the cancer and not-cancer samples still overlap. One biomarker simply cannot completely separate healthy from disease with a high degree of accuracy. The equation used in this example causes an inversion of the order of the concentration values when transitioned into a Proximity Score, in zones above and below the age adjusted mean values of concentration for cancer and not-cancer, respectively. There are two cases discussed here. The first case is where zones 1 and 2 are above the mean value for not-disease and below the midpoint; and where zones 3 and 4 are above the midpoint but below the mean value for disease. The second case is where the zones are staged sequentially on the Proximity Score axis, with the mean for not-disease placed between zones 1 and 2; the mean for disease placed between zones 3 and 4 and the derived midpoint between zones 2 and 3. The first case has been used in situations where the population distribution of the not-disease and disease are in disparity (e.g., breast cancer—not-breast cancer is 0.5% and 99.5%, respectively which reflects a Local Population Bias). The second case has been used where the population distribution is closer to the training set distribution (e.g., aggressive/non-aggressive prostate cancer).

Note that now the mean value age transitions for not-cancer, midpoint and cancer mean values are each a single vertical line at the ordinate axis. Also note that the very low and very high values are logarithmically compressed and the values near the age related mean values are expanded somewhat. On the inversion, it is important to note that keeping the linear order is not important in the proximity correlation method, simply the proximity relations must be maintained. In other words, the order can be inverted. The compression and expansion normalizes the grand or overall distribution of the data but the close in spatial relations are maintained. This is termed removing spatial bias. The method removes negative spatial bias and smearing of the data due to age or other physiological variables, e.g. body mass index. In essence, the training set sample data points are forced to take positions in one of the 4 zones: 1) below age related mean for not-cancer; 2) between age related mean for not-cancer and the midpoint transition to cancer; 3) above the midpoint transition and below the age related mean for cancer; and 4) above the age related mean for cancer regardless of age or spatial distribution non-linearities.

Note that several other equations could be used in this method as long as the spatial biased is dealt with. Simple log compression from low concentrations to the age related mean for not-cancer, and for high concentrations above the age related mean for cancer and perhaps a sigmoid equation between these mean values. It is not possible to a priori determine what equation relationships for this transition, and the best fit must be determined by experiment and comparison of results via overall multi-marker ROC curves. The best equation depends on the character of the spatial bias.

### Summary of Analytical Steps

1) Choose biomarkers that have a functional relation to the disease of interest. The fact that the biomarker may have very poor disease predictive power (poor ROC curve) cannot eliminate it for consideration as two poor biomarkers with a large independent action in the transition from not-disease to disease may produce a very large amplification of predictive power. These biomarkers should have a functional distinction on their actions.

2) Carefully define the disease and not-disease cohorts for the Training Set. These sets should mimic the population that the test will be administered to. Unrelated non-conditions unrelated to the disease should not be eliminated. Nonmalignant conditions that are within the population should be statistically correct for both the cancer and not-cancer cohorts.

3) Measure the mean values of concentration for each cohort with sufficient age sampling to accurately determine how the age affects the mean values.

4) Convert the raw concentration values into the Proximity Score. On a two axis plot, this transformation will encompass forcing all raw concentration values equal to or very near the respective mean values onto a fixed but different (separated) numerical values on the Proximity Score axis regardless and independent of the samples age. Also, the raw concentration values at or very near the calculated midpoint in concentration between the not-disease and disease mean values must be mathematically forced to a fixed value on the Proximity Score axis regardless of the samples age. The midpoint Proximity Score Point should be between the low not-disease (usually) and high disease fix points on the proximity Score axis. This location arrangement is usually desirable but may not always be (e.g., a biomarker that up regulates at low ages but down regulates at higher ages may require a different strategy for Proteomics Variance suppression).

5) Mathematically compress or expand (or other) the raw concentration data such that it lands in its proper place regarding its relationship to the mean values at it age (make the solders line up by rank). While applying the Spatial Proximity Correlations method, adjust or experiment with the mathematical schema to maximize predictive power with the training set group. There are not a priory rules and the mathematical schema that meets the diagnostic goals will change depending on the character, non-linearly and complexity of the raw measurement involved in the transition from not-disease to disease. The Complexity Paradox (Kenneth L. Mossman, Oxford University Press, 2014), the challenges faced by Proteomic Investigators are aptly summarized: “the non-linear dynamics inherent in complex biological systems leads to irregular and unpredictable behaviors.”

6) Use the exact same mathematical schema to compute disease scores on a test population that is equivalent to the target population for the test. Determine if this validation sample set meets diagnostic criterion.

### Predicting Tumor Status and Aggressiveness

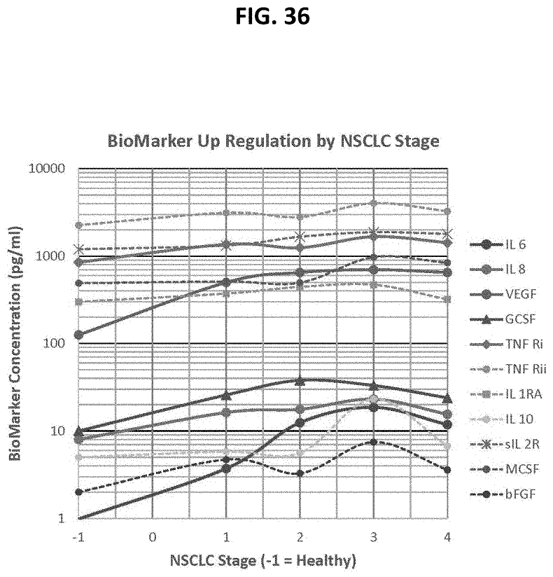

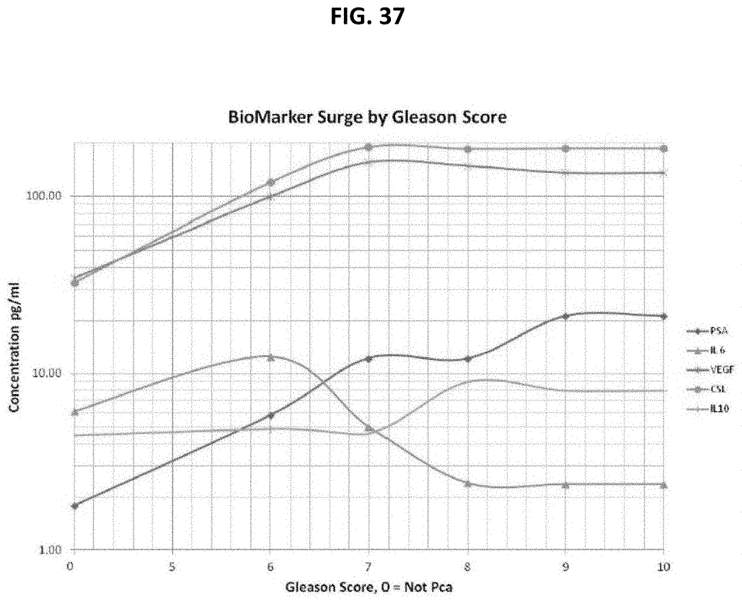

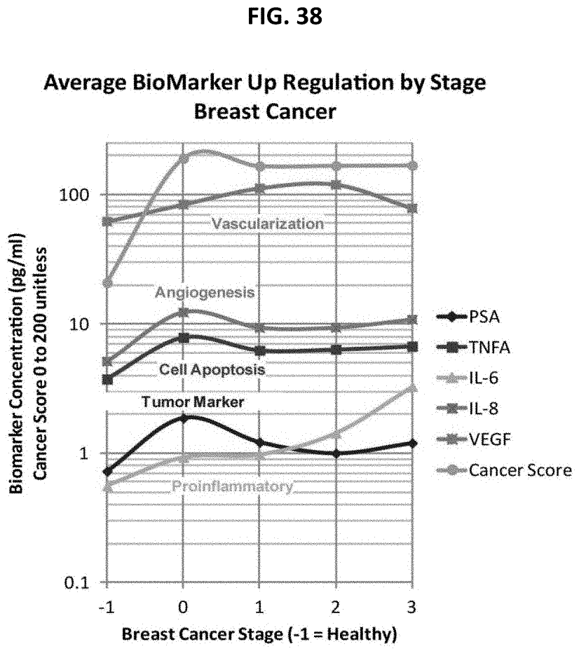

FIGS. 36, 37 and 38 show the actions of a number of different biomarkers as the tumor progresses for stage to later stage; in the case of prostate cancer Gleason Score is shown. These three graphs show similar behaviors for all three cancers for their respective TME active biomarkers. Note that in the early stage, the immune system reacts to the nascent tumor aggressively. Pro-inflammatory and anti-tumor genesis (apoptosis) biomarkers spike up. Typically, the angiogenesis response is also strong in the early tumor stage (see breast and NSCLC). The vascularization response of the tumor tends to increase as the tumor grows. Also, the tumor tends to secrete anti-inflammatory cytokines (TME active) to suppress the immune system in the later stages. That is especially true of aggressive prostate cancer (Gleason Score 8, 9 and 10).

This modulation of these TME active biomarkers allows, using a different training set model to call the current stage of the tumor. We have done this for breast and NSCLC cancer with 97% accuracy for both. In the case of prostate cancer, the transition from low grade or non-aggressive prostate cancer to the aggressive state can be predicted with 95% accuracy.

The spatial proximity correlation method produces a binary outcome prediction. The method will determine whether the unknown samples are either “State A” or “Not State A”. After determining the stage (or Gleason score for prostate cancer), the strategy must be modified. For the case where cancer stage or 0, 1, 2, 3 or 4 may exist, the strategy is to cluster the stages into sets of binary groups. Thus, for the case noted, the clusters of binary groups would be 1) stage 0 versus stages 1, 2, 3, 4; 2) stage 1, versus stage 0, 2, 3, 4; 3) stage 2, stage 0, 1, 3, 4; 4) stage 3 versus stage 0, 1, 2, 4; and 5) stage 4 versus stage 0, 1, 2, 3. These 5 clusters are then scored by the Spatial Proximity Correlation Method. The individual stage levels are then de-convoluted from the composite groups of models to produce the outright score for each stage. This method will produce the predictive power values noted above, 95% to 97%.

### Exemplary Methods

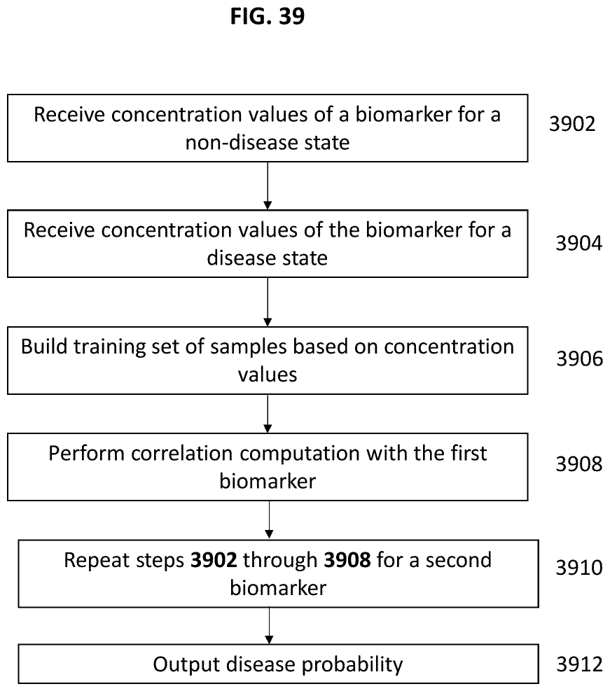

FIG. 39 shows an exemplary pathway by which the method of the present invention may be performed. The method commences at step 3902, “Receive concentration values of a biomarker for a non-disease state,” where the system receives an input of concentration values of a first biomarker from a first set of samples from patients with a not-disease diagnosis. Then, at step 3904, “Receive concentration values of the biomarker for a disease state,” the system receives an input of concentration values of a second biomarker from patients with a disease diagnosis. Then, at step 3906, “Build training set of samples based on concentration values,” the concentration values of the biomarker are used to build a training set of samples. At step 3908, “Perform correlation computation with the first biomarker,” the system computes a correlation computation for the first biomarker from the first set of concentration values combined with the concentration values of the first biomarker from the second set of concentration values for that biomarker. In various embodiments, that computation calculations may be simple regression, neural networks, ROC curve area maximization, random forest methods, support vector machine or other industry standard methods known in the art. At step 3910, “Repeat steps 3902 through 3908 for a second biomarker,” steps 3902 through 3908 are repeated for a second biomarker. While repeating those steps, the training set model of samples is updated to account for the combined effects on disease and non-disease state of the first and second biomarkers used in the analysis. In certain embodiments, the second biomarker is analyzed independently, while in others it is analyzed in conjunction with the first biomarker in a multi-dimensional space. In yet other embodiments, the second biomarker may be functionally orthogonal to the first biomarker. Having analyzed the first and second biomarkers as outlined exemplarily above, the system, at step 3912, “Output disease probability,” outputs a probability of disease state based on inputs that it receives for individual patients under examination with various concentrations of the two biomarkers. As noted above, that probability determination may be based on proximity scoring. In certain embodiment, the determination of disease probability may involve computation from the derived exclusion and inclusion zones, as well as the counting of set point values from the training set. The probability of a disease state is then based on the outputted score, which is reported by the system.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A computer-implemented method of creating an evaluative model that indicates a probability of a disease state in a patient under examination, the method comprising:
a. receiving a first set of concentration values of a first biomarker from a first set of samples from patients with a not-disease diagnosis;
b. receiving a second set of concentration values of the first biomarker from a second set of samples from patients with a disease diagnosis, wherein the first set and second set of samples comprise a training set of samples;
c. completing a correlation computation for the first biomarker from the first set of concentration values combined with the concentration values of the first biomarker from the second set of concentration values, wherein said computation may be simple regression, neural networks, ROC curve area maximization, random forest methods, support vector machine or other industry standard methods; and
d. performing steps (a) through (c) for a second biomarker wherein the second biomarker is functionally orthogonal to the first biomarker, and wherein the second biomarker is analyzed independently or in conjunction in a multi-dimensional space with the first biomarker to indicate the probability of a disease state.

2. The computer implemented method of claim 1, wherein the training set of samples includes at least one of blood samples, urine samples, and tissue samples.

3. The computer-implemented method of claim 1, wherein the training set of samples includes an equal number of disease samples and not-disease samples.

4. The computer implemented method of claim 3, wherein the disease being diagnosed is
a. non-small cell lung cancer; or
b. stages of non-small cell lung cancer segregated by stage.

5. The computer implemented method of claim 4, wherein the biomarkers are selected from functional groups of cytokines, where the functional groups are at least three of pro-inflammatory, antitumor genesis or cell apoptosis, angiogenesis, vascularization cytokine and colony stimulating factor functions.

6. The computer implemented method of claim 5, wherein one of the biomarkers is interleukin 6.

7. The computer implemented method of claim 5, wherein one of the biomarkers is vascular endothelial growth factor beta.

8. The computer implemented method of claim 5, wherein one of the colony stimulating factor functions is granulocyte-colony stimulating factor.

9. The computer implemented method of claim 5, wherein one of the pro-inflammatory factors is interleukin 1, interleukin 1β, IL-12, or IL-18.

10. The computer implemented method of claim 5, wherein one of the antitumor genesis or cell apoptosis factors is CD254, DR3L, CD258 or TNA receptors 2.

11. The computer implemented method of claim 5, wherein one of the vascularization factors is Placental Growth Factor (PLGF), VEGF-A, VEGF-C or VEGF-D.

12. The computer implemented method of claim 5, wherein one of the colony stimulating factors is GM-CSF or macrophage stimulating factor GSF.

13. The computer implemented method of claim 3, wherein the disease being diagnosed is stages of solid tumor cancers such as breast, ovarian, melanoma; and wherein a tumor marker specific to that cancer is added to the test.

14. The computer implemented method of claim 11, wherein the samples with stage information are grouped into binary groups with each stage represented on either one side of the binary set or the other grouped with the remaining stages.

15. The computer implemented method of claim 17, wherein all of the binary groupings of samples with cancer stage are scored.

16. The computer implemented method of claim 18, wherein each sample is scored individually by adding the score for the grouped binary groups with a weighting factor representing the fractional contribution to the score for that group.

17. The computer-implemented method of claim 1, wherein the training set of samples includes samples from patients within a predetermined range of ages.

18. The computer-implemented method of claim 1, wherein the disease diagnosis is selected from the group consisting of the stages of a cancer.

19. The computer-implemented method of claim 2, wherein the cancer is selected from the group consisting of breast cancer, renal cancer, ovarian cancer, lung cancer, melanoma and prostate cancer.

20. The computer-implemented method of claim 2, wherein the not-disease diagnosis includes four of the five stages, and the disease diagnosis includes the remaining stage.

21. A non-transitory computer-readable medium storing an evaluative model created by the method of claim 1 that indicates a probability of a disease state in a patient under examination.
